(19) United States
(12) Patent Application Publication
(10) Pub. No.: US 2024/0026448 A1
(43) Pub. Date: Jan. 25, 2024

(54) DETECTABLE PROBES AND COMPLEXES AND RELATED METHODS

(71) Applicant: 10x Genomics, Inc., Pleasanton, CA (US)

(72) Inventor: Justin COSTA, Union City, CA (US)

(21) Appl. No.: 18/204,264

(22) Filed: May 31, 2023

Related U.S. Application Data
(60) Provisional application No. 63/347,900, filed on Jun. 1, 2022.

(51) Int. Cl. C12Q 1/6876; C12Q 1/6813; G01N 21/35

(52) U.S. Cl. CPC C12Q 1/6876; C12Q 1/6813; G01N 21/35; C12Q 2600/16

(57) ABSTRACT

The present disclosure relates in some aspects to methods and compositions for in situ analysis using nucleic acid complexes comprising a detectable label and a moiety that can be activated to extinguish signals of the detectable label. In some embodiments, the activatable moiety comprises a photosensitizer. The nucleic acid complexes may allow for improved detection and decreased signal carryover between detection cycles.

### CROSS-REFERENCE TO RELATED APPLICATIONS

This application claims priority to U.S. Provisional Patent Application No. 63/347,900, filed Jun. 1, 2022, entitled “DETECTABLE PROBES AND COMPLEXES AND RELATED METHODS,” which is herein incorporated by reference in its entirety for all purposes.

### FIELD

The present disclosure relates in some aspects to methods and compositions for in situ analysis using probes or complexes comprising a detectable label and a photosensitizer.

### BACKGROUND

Methods are available for analyzing analytes such as nucleic acids present in a biological sample, e.g., a cell or tissue sample. Current methods for analyzing analytes in situ can have low sensitivity and specificity, have limited plexity, or be biased, time-consuming, labor-intensive, and/or error-prone. Improved methods for analyzing analytes in a biological sample are needed. Provided herein are methods and compositions that meet such and other needs.

### SUMMARY

Analyzing analytes (e.g., nucleic acids or proteins) in a biological sample in situ may involve use of detectable probes, e.g., detectably labeled oligonucleotides comprising a signal producing detectable label and/or probes capable of hybridizing to detectably labeled oligonucleotides. Inefficient removal of the signal between rounds of probe binding and signal detection may complicate the optical readout for decoding the analytes. In some aspects, provided herein are probes and complexes that facilitate efficient removal of signals and/or signal producing detectable labels and reduce signal carryover from cycle to cycle. In some embodiments, the probes and complexes disclosed herein are armed with one or more photosensitizers. In some embodiments, the probes and complexes disclosed herein have one or more other functional moieties such as affinity moieties.

In some aspects, provided herein is a method for analyzing a biological sample, comprising: a) forming a nucleic acid complex in the biological sample, wherein the biological sample comprises a target nucleic acid comprising a target sequence, and wherein the nucleic acid complex comprises a detectable label, a photosensitizer, and a target-binding region hybridized to the target sequence; b) detecting a signal associated with the detectable label of the nucleic acid complex; and c) activating the photosensitizer of the nucleic acid complex, thereby attenuating the signal associated with the detectable label. In some embodiments, the detectable label and the photosensitizer are in proximity in the nucleic acid complex.

In any of the embodiments herein, the nucleic acid complex can comprise one, two, or more nucleic acid molecules. In any of the embodiments herein, the nucleic acid complex can comprise a nucleic acid probe and a splint oligonucleotide hybridized thereto, where the nucleic acid probe comprises the target-binding region. In some embodiments, the nucleic acid probe comprises the photosensitizer and the splint oligonucleotide comprises the detectable label. In some embodiments, the nucleic acid probe comprises the detectable label and the splint oligonucleotide comprises the photosensitizer.

In any of the embodiments herein, the nucleic acid complex can further comprise a coupling oligonucleotide that hybridizes to the splint oligonucleotide and/or the nucleic acid probe. In some embodiments, the nucleic acid complex comprises two or more coupling oligonucleotides hybridized to the splint oligonucleotide and/or the nucleic acid probe. In any of the embodiments herein, the splint oligonucleotide can comprise: i) a hybridization region that hybridizes to a complementary hybridization region in the coupling oligonucleotide, and ii) a hybridization region that hybridizes to a complementary hybridization region in the nucleic acid probe. In any of the embodiments herein, the splint oligonucleotide can comprise the detectable label and the coupling oligonucleotide can comprise the photosensitizer. In some embodiments, the splint oligonucleotide comprises the photosensitizer and the coupling oligonucleotide comprises the detectable label. In any of the embodiments herein, the splint oligonucleotide and the coupling oligonucleotide can be configured to covalently and/or noncovalently couple to each other. In any of the embodiments herein, the splint oligonucleotide and the coupling oligonucleotide can be configured to covalently couple to each other.

In any of the embodiments herein, the nucleic acid probe, the splint oligonucleotide, and/or the coupling oligonucleotide can comprise one, two, or more nucleic acid molecules that are covalently and/or non-covalently attached to one another.

In some embodiments, the splint oligonucleotide and/or the coupling oligonucleotide each independently comprises a photoreactive nucleotide capable of reacting with a nucleotide in an oligonucleotide strand hybridized thereto to form a covalent bond. In some embodiments, the photoreactive nucleotide comprises a 3-cyanovinylcarbazole (^{CNV}K) nucleoside, 3-cyanovinylcarbazole modified D-threoninol (^{CNV}D), or 3-cyanovinylcarbazole phosphoramidite.

In any of the embodiments herein, the method can comprise photo-activating the photoreactive nucleotide to covalently couple the splint oligonucleotide and the coupling oligonucleotide. In some embodiments, the photo-activating is performed using a light having a wavelength of 350-400 nm. In any of the embodiments herein, the photo-activating can be performed prior to the splint oligonucleotide and the coupling oligonucleotide contacting the biological sample. In any of the embodiments herein, the photo-activating can be performed without activating the photosensitizer.

In any of the embodiments herein, the splint oligonucleotide can comprise the detectable label, and the method can comprise: i) contacting the splint oligonucleotide and the coupling oligonucleotide with an oligonucleotide comprising the photosensitizer, and ii) ligating the oligonucleotide comprising the photosensitizer to the coupling oligonucleotide using the splint oligonucleotide as a template, whereby the photosensitizer and the detectable label are brought into proximity with each other. In some embodiments, the splint oligonucleotide and the coupling oligonucleotide are covalently coupled to each other prior to or after the ligation.

In any of the embodiments herein, any two or all of the nucleic acid probe, the splint oligonucleotide, and the coupling oligonucleotide can form a complex prior to contacting with the biological sample. In some embodiments, the splint oligonucleotide and the coupling oligonucleotide form a complex outside the biological sample prior to the complex contacting the biological sample. In some embodiments, the nucleic acid probe and the complex are contacted with the biological sample simultaneously or sequentially. In some embodiments, the nucleic acid probe is hybridized to the target nucleic acid in the biological sample prior to the complex contacting the biological sample. In some embodiments, the nucleic acid probe, the splint oligonucleotide, and the coupling oligonucleotide form a complex outside the biological sample prior to the complex contacting the biological sample.

In any of the embodiments herein, any two or all of the nucleic acid probe, the splint oligonucleotide, and the coupling oligonucleotide can form a complex in the biological sample. In some embodiments, the nucleic acid probe and the splint oligonucleotide form a complex in the biological sample prior to the coupling oligonucleotide contacting the biological sample.

In any of the embodiments herein, the signal associated with the detectable label can be detected without activating the photosensitizer in the nucleic acid complex.

In any of the embodiments herein, the photosensitizer can be activated using an infrared light. In any of the embodiments herein, the photosensitizer can be activated using a light having a wavelength between about 800 nm and about 1 mm. In any of the embodiments herein, the photosensitizer can be activated using a light having a wavelength between about 800 nm and about 2,500 nm. In any of the embodiments herein, the photosensitizer can comprise phthalocyanine or a derivative thereof, Atto-Thio12, an organometallic photosensitizer, Methylene Blue, or Rose Bengal. In some embodiments, the organometallic photosensitizer is Tris(2-phenylpyridine)iridium.

In any of the embodiments herein, activation of the photosensitizer can lead to oxidation of the detectable label. In any of the embodiments herein, activation of the photosensitizer can generate a reactive oxygen species. In some embodiments, the reactive oxygen species comprises a free radical and/or a non-radical. In some embodiments, the reactive oxygen species comprises O^{•-}₂, OH^{•}, H₂O₂, and/or singlet oxygen (¹O₂).

In some embodiments, the reactive oxygen species reacts with the detectable label to render it undetectable under the conditions for the detecting the signal associated with the detectable label. In any of the embodiments herein, the reactive oxygen species can permanently extinguish the signal associated with the detectable label.

In any of the embodiments herein, the reactive oxygen species can decay to below 50%, below 10%, or below 1% within 0.1 nm, 1 nm, 5 nm, 10 nm, 20 nm, 25 nm, 30 nm, 35 nm, 40 nm, 45 nm, or 50 nm from the photosensitizer.

In any of the embodiments herein, the detectable label can comprise a luminophore. In some embodiments, the luminophore is a fluorophore.

In any of the embodiments herein, the nucleic acid complex can further comprise an affinity moiety. In some embodiments, the affinity moiety is covalently coupled to the nucleic acid complex via a covalent bond or a linker. In any of the embodiments herein, the nucleic acid probe, the splint oligonucleotide, and/or the coupling oligonucleotide can each independently comprise one, two, or more affinity moieties. In any of the embodiments herein, the affinity moiety can comprise a biotin or derivative thereof. In any of the embodiments herein, the method can comprise contacting the biological sample with a binding partner of the affinity moiety after the signal associated with the detectable label is detected. In some embodiments, the binding partner is coupled to a nanoparticle. In any of the embodiments herein, the binding partner can be coupled to a diamagnetic, paramagnetic, or ferromagnetic particle. In any of the embodiments herein, the method can comprise removing a complex comprising the binding partner and the nucleic acid probe, the splint oligonucleotide, and/or the coupling oligonucleotide from the biological sample.

In any of the embodiments herein, the nucleic acid complex can further comprise a reflective moiety. In some embodiments, the reflective moiety is covalently coupled to the nucleic acid complex via a covalent bond or a linker. In any of the embodiments herein, the nucleic acid probe, the splint oligonucleotide, and/or the coupling oligonucleotide can each independently comprise one, two, or more reflective moieties. In any of the embodiments herein, the reflective moiety can reflect infrared light. In any of the embodiments herein, the reflective moiety can comprise a gold nanoparticle.

In any of the embodiments herein, the signal associated with the detectable label can be detected in situ and the photosensitizer is activated in situ.

In any of the embodiments herein, the target nucleic acid can comprise or can be a cellular nucleic acid molecule or a product thereof. In some embodiments, the cellular nucleic acid molecule or product thereof is a genomic DNA, mRNA, or cDNA.

In any of the embodiments herein, the target nucleic acid can comprise or can be a primary probe that hybridizes to a cellular nucleic acid molecule or a product thereof. In some embodiments, the primary probe is selected from the group consisting of: a primary probe comprising a 3′ or 5′ overhang upon hybridization to the cellular nucleic acid molecule or product thereof, a primary probe comprising a 3′ overhang and a 5′ overhang upon hybridization to the cellular nucleic acid molecule or product thereof; a circular primary probe; a circularizable primary probe or probe set; a primary probe or probe set comprising a split hybridization region configured to hybridize to a splint; and a combination thereof. In some embodiments, the 3′ overhang and the 5′ overhang of the primary probe each independently comprises one or more barcode sequences. In some embodiments, the target-binding region hybridizes to the one or more barcode sequences in the primary probe. In some embodiments, the one or more barcode sequences comprise a split barcode sequence.

In any of the embodiments herein, the target nucleic acid can comprise or be an intermediate probe that hybridizes to a primary probe or a product or complex thereof, wherein the primary probe hybridizes to a cellular nucleic acid molecule or a product thereof In some embodiments, the product or complex of the primary probe is selected from the group consisting of: a rolling circle amplification (RCA) product; a complex comprising an initiator and an amplifier for hybridization chain reaction (HCR); a complex comprising an initiator and an amplifier for linear oligonucleotide hybridization chain reaction (LO-HCR); a primer exchange reaction (PER) product; and a complex comprising a pre-amplifier and an amplifier for branched DNA (bDNA). In any of the embodiments herein, the intermediate probe can be selected from the group consisting of: an intermediate probe comprising a 3′ or 5′ overhang upon hybridization to the primary probe or product or complex thereof, an intermediate probe comprising a 3′ overhang and a 5′ overhang upon hybridization to the primary probe or product or complex thereof, a circular intermediate probe; a circularizable intermediate probe or probe set; an intermediate probe or probe set comprising a split hybridization region configured to hybridize to a splint; and a combination thereof. In some embodiments, the intermediate probe hybridizes to one or more barcode sequences in the primary probe or product or complex thereof.

In any of the embodiments herein, the target nucleic acid can comprise or be a reporter oligonucleotide of a labelling agent, the labelling agent comprising an analyte-binding region and the reporter oligonucleotide. In some embodiments, the analyte comprises a nucleic acid, a protein, a carbohydrate, a lipid, or a small molecule, or a complex thereof.

In any of the embodiments herein, the target nucleic acid can comprise an overhang region comprising multiple copies of the target sequence; or the target nucleic acid can be concatemeric and comprise multiple copies of the target sequence.

In some embodiments, the target nucleic acid comprises a rolling circle amplification (RCA) product of a circular or circularized probe that hybridizes to a nucleic acid molecule in the biological sample, wherein the circular or circularized probe comprises a barcode region.

In any of the embodiments herein, the method can further comprise generating the target nucleic acid or a molecule or complex to which the target nucleic acid directly or indirectly binds. In some embodiments, the target nucleic acid or the molecule or complex is generated in situ in the biological sample. In some embodiments, generating the target nucleic acid or the molecule or complex comprises by (i) contacting the biological sample with a circular or circularizable probe or probe set that hybridizes to a nucleic acid molecule in the biological sample; and (ii) performing a rolling circle amplification (RCA) using the circular probe or a circularized probe as template, wherein the circularized probe is generated by circularizing the circularizable probe or probe set.

In any of the embodiments herein, the nucleic acid complex can be a first nucleic acid complex, the target nucleic acid can be a first target nucleic acid, the target sequence can be a first target sequence, the detectable label can be a first detectable label, the photosensitizer can be a first photosensitizer, and the method can further comprise: a′) forming a second nucleic acid complex in the biological sample, wherein the biological sample comprises a second target nucleic acid comprising a second target sequence, and wherein the second nucleic acid complex comprises a second detectable label, a second photosensitizer, and a second target-binding region hybridized to the second target sequence; b′) detecting a second signal associated with the second detectable label of the second nucleic acid complex; and c′) activating the second photosensitizer of the second nucleic acid complex, thereby attenuating the second signal. In some embodiments, the first target nucleic acid and the second target nucleic acid are the same or different, and wherein the first target sequence and the second target sequence are the same or different. In any of the embodiments herein, the first detectable label and the second detectable label can be the same or different. In any of the embodiments herein, the first photosensitizer and the second photosensitizer can be the same or different. In any of the embodiments herein, the first target sequence and the second target sequence can be the same, and the first photosensitizer and the second photosensitizer can be the same.

In some embodiments, provided herein is a method for analyzing a biological sample, comprising: a) generating a rolling circle amplification (RCA) product in the biological sample, the RCA product comprising multiple copies of a barcode sequence, wherein the barcode sequence is associated with an analyte of interest and is assigned a signal code sequence; b) contacting the biological sample with a first probe and a first probe complex (e.g., as shown in FIG. 3) to generate a first nucleic acid complex comprising the first probe hybridized to the RCA product and the first probe complex hybridized to the first probe, wherein the first probe comprises (i) a recognition sequence complementary to the barcode sequence and (ii) a first overhang sequence, and wherein the first probe complex comprises a first splint oligonucleotide and a first coupling oligonucleotide hybridized thereto, wherein the first splint oligonucleotide comprises a first detectable label and a sequence complementary to the first overhang sequence, and wherein the first coupling oligonucleotide comprises a first photosensitizer; c) detecting a first signal associated with the first detectable label, wherein the first signal corresponds to a first signal code in the signal code sequence; d) activating the first photosensitizer, thereby attenuating the first signal; e) contacting the biological sample with a second probe and a second probe complex (e.g., as shown in FIG. 3) to generate a second nucleic acid complex comprising the second probe hybridized to the RCA product and the second probe complex hybridized to the second probe, wherein the second probe comprises (i) a recognition sequence complementary to the barcode sequence and (ii) a second overhang sequence, and wherein the second probe complex comprises a second splint oligonucleotide and a second coupling oligonucleotide hybridized thereto, wherein the second splint oligonucleotide comprises a second detectable label and a sequence complementary to the second overhang sequence, and wherein the second coupling oligonucleotide comprises a second photosensitizer; and f) detecting a second signal associated with the second detectable label, wherein the second signal corresponds to a second signal code in the signal code sequence, wherein the signal code sequence comprising the first signal code and the second signal code is determined at a location in the biological sample, thereby decoding the barcode sequence and identifying the analyte of interest at the location in the biological sample. In some embodiments, the method can further comprise activating the second photosensitizer, thereby attenuating the second signal.

In any of the embodiments herein, the first probe, the second probe, and one or more subsequent probes can be contacted with the biological sample sequentially in a pre-determined sequence which corresponds to the signal code sequence assigned to the barcode sequence, wherein the one or more subsequent probes can each comprise (i) a recognition sequence complementary to the barcode sequence and (ii) an overhang sequence complementary to a probe complex of a universal pool of probe complexes. In some embodiments, the first probe complex and the second probe complex are in the universal pool of probe complexes.

In any of the embodiments herein, the contacting of the biological sample with a first probe and a first probe complex can comprise contacting the biological sample with the universal pool of probe complexes, and the contacting of the biological sample with a second probe and a second probe complex comprises contacting the biological sample with the universal pool of probe complexes. In any of the embodiments herein, the one or more subsequent probes can be contacted with the biological sample to determine signal codes in the signal code sequence until sufficient signal codes have been determined to decode the barcode sequence, thereby identifying the target analyte. In any of the embodiments herein, the method can further comprise between the activating of the first photosensitizer and the contacting of the biological sample with a second probe and a second probe complex, a step of removing the first probe and/or the first probe complex from the biological sample, and/or after the detecting step the second signal associated with the second detectable label, a step of removing the second probe and/or the second probe complex from the biological sample, before contacting the sample with a subsequent probe.

In any of the embodiments herein, the barcode sequence associated with the target analyte can be selected from a plurality of barcode sequences, and wherein the contacting of the biological sample with a first probe and a first probe complex can comprise contacting the sample with a first pool of probes and a universal pool of probe complexes, wherein the first pool of probes can comprise the first probe and the universal pool of probe complexes can comprise the first probe complex and the second probe complex, wherein each probe in the first pool of probes can comprise (i) a recognition sequence complementary to one of the plurality of barcode sequences and (ii) an overhang sequence complementary to a probe complex of the universal pool of probe complexes; and wherein the contacting of the biological sample with a second probe and a second probe complex can comprise contacting the biological sample with a second pool of probes and the universal pool of probe complexes, wherein the second pool of probes can comprise the second probe, and wherein each probe in the second pool of probes can comprise (i) a recognition sequence complementary to one of the plurality of barcode sequences and (ii) an overhang sequence complementary to a probe complex of the universal pool of probe complexes.

In some embodiments, provided herein is a method for identifying multiple different target analytes present at locations in the biological sample, wherein each different target analyte is assigned a different signal code sequence and is targeted by a circularizable probe or probe set comprising a complement of a different barcode sequence of the plurality of barcode sequences. In any of the embodiments herein, the number of different probes in each pool of probes is greater than the number of different probe complexes in the universal pool of probe complexes. In any of the embodiments herein, the number of different probe complexes in the universal pool of probe complexes can be four. In any of the embodiments herein, the number of different probes in each pool of probes can be 50, 100, 200, 500, 1000, or more. In any of the embodiments herein, for each nucleic acid complex, the hybridization between the probe and the splint oligonucleotide can have a higher melting temperature than the hybridization between the probe and the RCA product.

In some embodiments, provided herein is a kit comprising a plurality of probe complexes, wherein each probe complex comprises a splint oligonucleotide and a coupling oligonucleotide hybridized thereto, the splint oligonucleotide comprises a detectable label, the coupling oligonucleotide comprises a photosensitizer, and the detectable label and the photosensitizer are in proximity in the probe complex. In some embodiments, each probe complex comprises the same photosensitizer and one of two, three, four, or more different detectable labels. In any of the embodiments herein, the kit can further comprise multiple probes, wherein each probe comprises i) a target-binding region complementary to a target sequence, and ii) an overhang sequence complementary to a splint oligonucleotide of the plurality of probe complexes. In some embodiments, the hybridization between the probe and the splint oligonucleotide has a higher melting temperature than the hybridization between the probe and the target sequence. In any of the embodiments herein, the target sequence comprises a barcode sequence or complement thereof corresponding to an analyte of interest. In any of the embodiments herein, the kit can comprise: a universal pool of probe complexes collectively comprising four different detectable labels each corresponding to one of four different splint oligonucleotides; the multiple probes collectively comprise at least or about 10, at least or about 25, at least or about 50, at least or about 100, at least or about 250, at least or about 500, at least or about 750, at least or about 1,000, or more different target-binding regions; and the multiple probes collectively comprise four different overhang sequences each of which is complementary to one of the four different splint oligonucleotides.

### BRIEF DESCRIPTION OF THE DRAWINGS

The drawings illustrate certain features and advantages of this disclosure. These embodiments are not intended to limit the scope of the appended claims in any manner.

FIGS. 1A-1F depict exemplary nucleic acid complexes and components thereof, and methods of generating probe complexes each comprising one or more coupling oligonucleotides and a splint oligonucleotide coupled together. The nucleic acid complex can comprise a detectable label, a photosensitizer, and a target-binding region configured to hybridize to a target sequence. Optional covalent crosslinks (e.g., generated via ^{CNV}K, ^{CNV}D, or 3-cyanovinylcarbazole phosphoramidite) are indicated by “X” in FIGS. 1E and 1F.

FIG. 2 depicts in situ hybridization of exemplary nucleic acid complexes to target nucleic acids in a cell or tissue sample. The nucleic acid complexes can be pre-formed prior to contacting the cell or tissue sample.

FIG. 3 depicts exemplary libraries of nucleic acid complexes comprising barcoded nucleic acid probes and in situ hybridization of the multiple libraries in sequential cycles to decode the barcodes in the nucleic acid probes and detect the analytes that the barcodes correspond to. Optional covalent crosslinks (e.g., generated via ^{CNV}K, ^{CNV}D, or 3-cyanovinylcarbazole phosphoramidite) are indicated by “X.”

FIG. 4 is an example workflow of analysis of a biological sample (e.g., a cell or tissue sample) using an opto-fluidic instrument, according to various embodiments.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

During in situ analysis, the efficient removal of a detectable signal (e.g., a fluorescent signal) from a previous detection step is crucial for proper signal detection. This can be especially important in methods involving detecting probe binding in sequential cycles and decoding of the sequential probe signals in order to identify analytes in a sample. For instance, detectable probes can be sequentially applied to a cell or tissue sample to combinatorially detect identifier sequences in a target nucleic acid. The identifier sequences can be nucleic acid analyte sequences or barcode sequences, which are not removed from the sample, whereas signals from a previous cycle are extinguished and/or the detectable probes are removed prior to contacting the sample with detectable probes for a subsequent cycle. However, signal may carryover between cycles. Undesirable carryover of signal may be due to pan-sample photo-damage, e.g., reactive singlet oxygen may crosslink detectably labeled probes (e.g., fluorescently labeled detection oligonucleotides) or detectable labels to the target nucleic acid and/or other molecules bound thereto. For example, in cases where rolling circle amplification (RCA) products comprising barcode sequences are detected, pan-sample photo-damage may crosslink fluorescent detection oligonucleotides to the RCA products, intermediate probes (e.g., L-probes), and/or other nearby molecules in the sample, thereby causing signal carryover even when the sample is washed under stringent conditions between cycles. Incomplete removal of intermediate probes (e.g., L-probes) that can later re-hybridize with their corresponding target sequences and/or fluorescent detection oligonucleotides in subsequent detection cycles may also contribute to signal carryover. Regardless of the cause, signal carryover may complicate the optical readout between detection cycles, and can therefore be highly problematic for decoding.

Detectably labeled probes (e.g., detection oligonucleotides comprising fluorescent labels) present a unique opportunity for optimization of in situ analysis, as they are usually the last reagent contacted with the biological sample prior to imaging, and therefore have a low risk of affecting or requiring changes in upstream aspects involved with in situ detection. In some embodiments, particularly in cases where a small number of different detectably labeled probes are used for decoding (e.g., four different detection oligonucleotides each for one of four distinct colors), various chemical functionalities can be reasonably achieved with low cost of goods and ease of manufacturing. Such complexities may not be practical to achieve in barcoded probes, such as barcoded primary probes that bind to target sequences in the biological sample, barcoded intermediate probes (e.g., L-probes), or barcoded circularizable probes used to generate RCA products.

In some aspects, the present disclosure provides nucleic acid complexes for the detection of target sequences of target nucleic acids in situ in a biological sample. Using the nucleic acid complexes may eliminate punctate nuclear background, for example, by activating an activatable moiety (e.g., a photosensitizer) to attenuate a signal of a nearby detectable label after the signal is detected and/or by facilitating physical removal of the detectable label along with the nucleic acid molecule or complex containing the detectable label. In some embodiments, using the nucleic acid complexes can help reduce residual background noise across the sequential detection cycles.

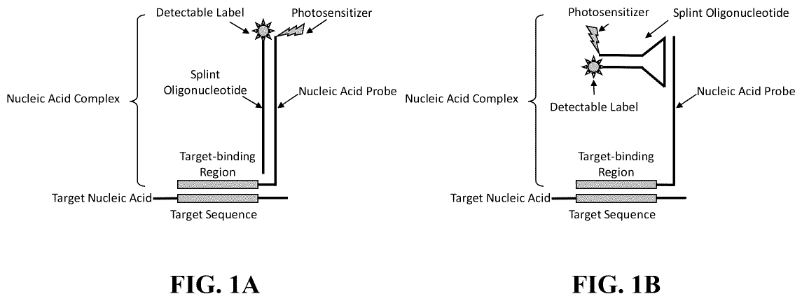

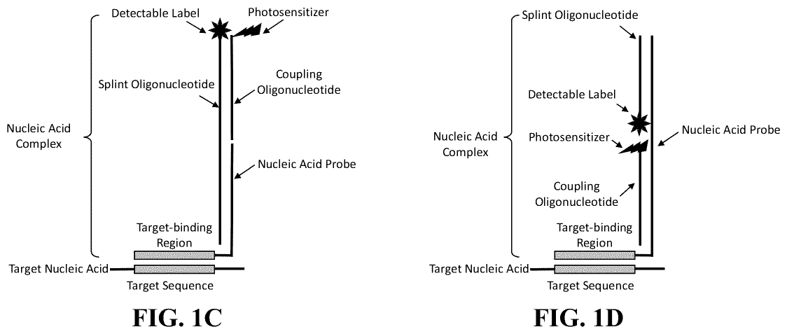

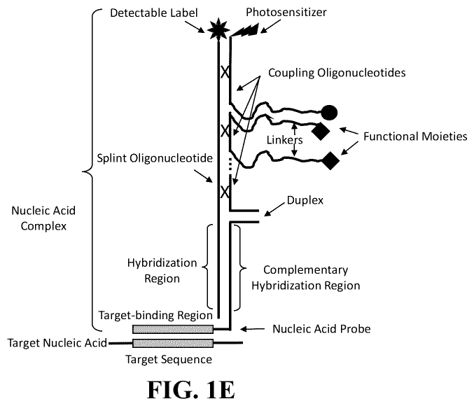

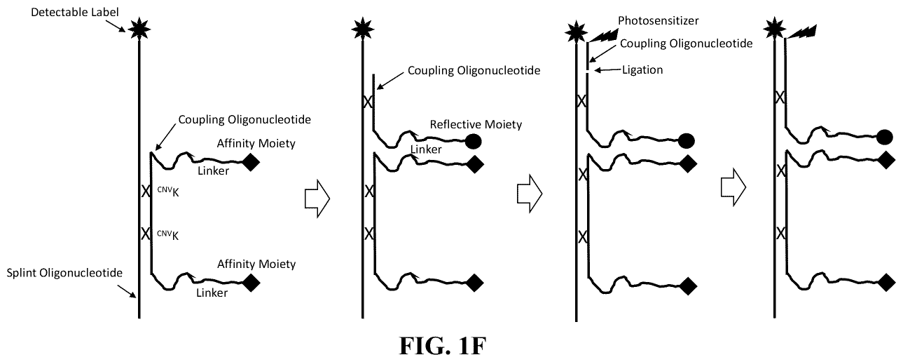

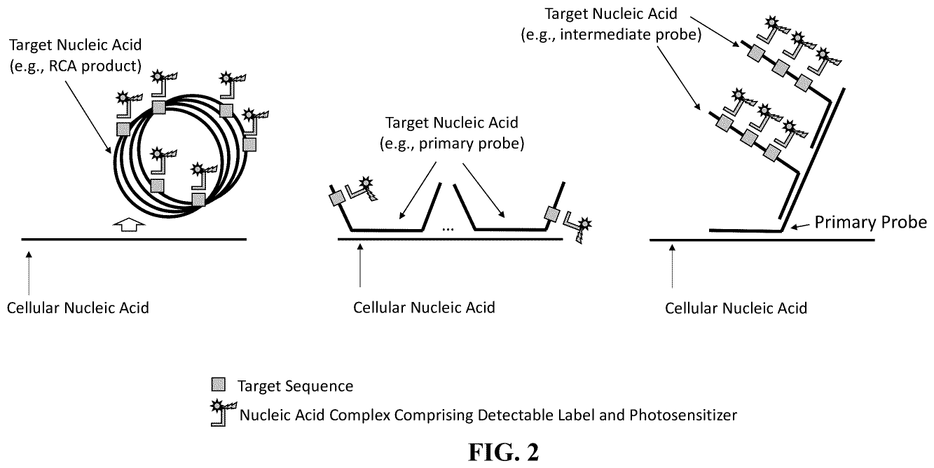

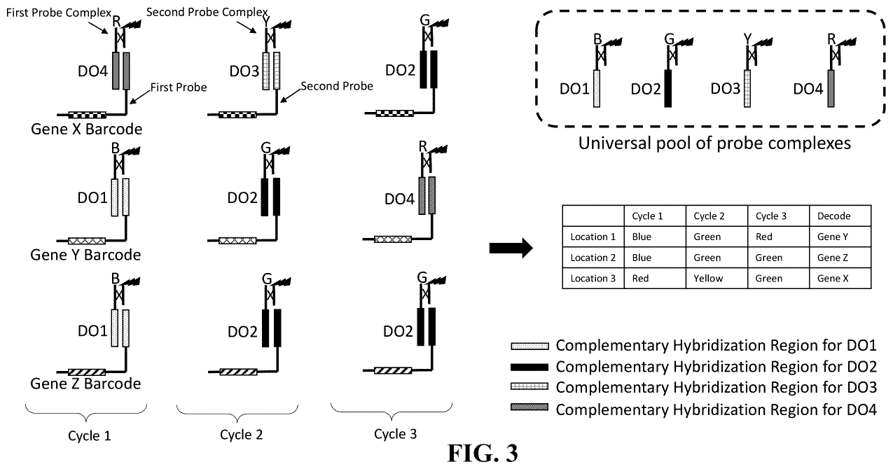

In some embodiments, provided herein is a method for analyzing a biological sample, comprising: a) forming a nucleic acid complex in the biological sample, wherein the biological sample comprises a target nucleic acid comprising a target sequence, and wherein the nucleic acid complex comprises a detectable label, a photosensitizer, and a target-binding region hybridized to the target sequence; b) detecting a signal associated with the detectable label of the nucleic acid complex; and c) activating the photosensitizer of the nucleic acid complex, thereby attenuating the signal associated with the detectable label. The nucleic acid complex may comprise one, two, or more nucleic acid molecules, such as a nucleic acid probe, a splint oligonucleotide, and/or a coupling oligonucleotide, e.g., as shown in FIGS. 1A-1E. The nucleic acid complex may be used to detect any suitable target nucleic acid, such as RCA products of a circularizable probe or nucleic acid analyte, primary probes the hybridize to cellular nucleic acids, or intermediate probes that hybridize to other probes (e.g., primary probes) or products of other probes. Exemplary target nucleic acids with nucleic acid complexes hybridized thereon are shown in FIG. 2. Multiple libraries of nucleic acid complexes targeting different analytes can be designed, as shown in FIG. 3, and each library of nucleic acid complexes can be contacted with a cell or tissue sample in a cycle. Signals associated with the library of nucleic acid complexes can be detected at multiple locations in the sample, after which the signals can be attenuated by activating the photosensitizers in the nucleic acid complexes and the nucleic acid complexes or portions thereof comprising the detectable labels can be optionally removed from the sample. In some embodiments, a new library of nucleic acid complexes are contacted with the sample after the signal detection, attenuation, and optional probe removal, and the cycles can be repeated as shown in FIG. 3 to decode multiple analytes at multiple locations in the sample. Attenuating of the signal associated with the detectable label using the photosensitizer in the same nucleic acid complex may allow for decrease background noise and decrease punctate nuclear background during subsequent rounds of detection.

### II. Methods for Analyzing a Biological Sample Using a Nucleic Acid Complex

Described herein are methods for analyzing a biological sample involving forming a nucleic acid complex in the biological sample. Also provided herein are multiplexing embodiments of analyzing a biological sample, comprising forming one or more nucleic acid complexes in the biological sample. The nucleic acid complex comprises a detectable label, a controllably activatable moiety (e.g., a photosensitizer), and a target-binding region capable of hybridizing to a target sequence in a target nucleic acid in the biological sample. In some embodiments, the controllably activatable moiety is photoactivatable. In some embodiments, the controllably activatable moiety is activated after a signal from the detectable label is detected, where the activated activatable moiety attenuates the signal. For instance, a signal associated with the detectable label of the nucleic acid complex may be detected, followed by activation of the photosensitizer of the nucleic acid complex, thereby attenuating the signal associated with the detectable label. The methods provided herein may be used to improve in situ decoding via efficient removal of fluorescent signal between detection cycles using a photosensitizer to attenuate the signal produced from the detectable label of a nucleic acid complex. Optionally, the signal may be further attenuated using one or more functional moieties such as affinity moieties (e.g., biotin moieties), which physically removes the detectable label (e.g., the probe comprising the detectable label, optionally the probe comprising the detectable label and other probes associated therewith) from the biological sample.

In some embodiments, provided herein is a nucleic acid complex comprising a detectable label, a photosensitizer, and a target-binding region complementary to a target sequence in a target nucleic acid. Any two or more of the detectable label, the photosensitizer, and/or the target-binding region of the nucleic acid complex can be in the same molecule or in different molecules. In some embodiments, the nucleic acid complex is pre-formed prior to contacting a sample, and the pre-formed nucleic acid complex may be formed via a covalent linkage (e.g., a covalent bond or linker) between two or more nucleic acid molecules and/or non-covalent interactions (e.g., based on nucleic acid sequence complementarity). In some embodiments, at least a portion of the nucleic acid complex can be formed in situ in the sample whereas at least another portion of the nucleic acid complex is pre-formed prior to contacting the sample.

For example, as shown in FIG. 1A, the nucleic acid probe can comprise the target-binding region and the photosensitizer, while the splint oligonucleotide can comprise the detectable label. The target-binding region can be at or near the 3′ end of the nucleic acid probe, the photosensitizer can be at or near (e.g., within about 10, about 8, about 6, about 4, or about 2 nucleotide residues from a terminal nucleic acid residue) the 5′ end of the nucleic acid probe, and the detectable label can be at the 3′ end of the splint oligonucleotide. Alternatively, the target-binding region can be at or near the 5′ end of the nucleic acid probe, the photosensitizer can be at or near the 3′ end of the nucleic acid probe, and the detectable label can be at the 5′ end of the splint oligonucleotide. In some embodiments, the splint oligonucleotide can comprise a hybridization region that hybridizes to a complementary hybridization region in the nucleic acid probe, and hybridization of the splint oligonucleotide and the nucleic acid probe brings the detectable label and the photosensitizer in physical proximity, such that activation of the photosensitizer can attenuate the signal from the detectable label (e.g., via oxidizing the detectable label) while minimizing damage or crosslinking of other nearby molecules in the sample. The nucleic acid complex in FIG. 1A can be pre-formed via sequence complementarity and/or a covalent linkage (e.g., a covalent bond or linker) between the splint oligonucleotide and the nucleic acid probe. Alternatively, the splint oligonucleotide and the nucleic acid probe can be separately contacted with the sample, either simultaneously or sequentially in either order, and the nucleic acid complex can be formed in situ in the sample.

FIG. 1B shows an example where the nucleic acid probe comprises the target-binding region, and the splint oligonucleotide comprises the detectable label and the photosensitizer. The photosensitizer and the detectable label can be at or near the ends of the splint oligonucleotide, and the splint oligonucleotide comprises a hybridization region that hybridizes to a complementary hybridization region in the nucleic acid probe. In some embodiments, intra-molecular hybridization of the splint oligonucleotide brings the detectable label and the photosensitizer in physical proximity, such that activation of the photosensitizer can attenuate the signal from the detectable label (e.g., via oxidizing the detectable label) while minimizing damage or crosslinking of other nearby molecules in the sample. The nucleic acid complex in FIG. 1B can be pre-formed via sequence complementarity and/or a covalent linkage (e.g., a covalent bond or linker) between the splint oligonucleotide and the nucleic acid probe. Alternatively, the splint oligonucleotide and the nucleic acid probe can be separately contacted with the sample, either simultaneously or sequentially in either order, and the nucleic acid complex can be formed in situ in the sample.

FIG. 1C shows an example where the nucleic acid probe comprises the target-binding region, the coupling oligonucleotide comprises the photosensitizer, and the splint oligonucleotide comprises the detectable label. The photosensitizer and the detectable label can be at or near an end of the coupling oligonucleotide or the splint oligonucleotide, respectively. The splint oligonucleotide can comprise: i) a first hybridization region that hybridizes to a first complementary hybridization region in the coupling oligonucleotide, and ii) a second hybridization region that hybridizes to a second complementary hybridization region in the nucleic acid probe. In some embodiments, the first hybridization region is 3′ to the second hybridization region. In some embodiments, the first hybridization region is 5′ to the second hybridization region. In some embodiments, the first and second hybridization regions do not overlap. In some embodiments, the first and second hybridization regions are directly linked by a phosphodiester bond. In some embodiments, the first and second hybridization regions are linked by one or more nucleic acid residues. In some embodiments, hybridization of the splint oligonucleotide and the coupling oligonucleotide brings the detectable label and the photosensitizer in physical proximity, such that activation of the photosensitizer can attenuate the signal from the detectable label (e.g., via oxidizing the detectable label) while minimizing damage or crosslinking of other nearby molecules in the sample. The splint oligonucleotide and the coupling oligonucleotide in FIG. 1C can be pre-coupled via sequence complementarity and/or a covalent linkage (e.g., a covalent bond or linker), and the pre-coupled oligonucleotides and the nucleic acid probe can be separately contacted with the sample, either simultaneously or sequentially in either order, so that the nucleic acid complex can be formed in situ in the sample. In some embodiments, the splint oligonucleotide, the coupling oligonucleotide, and the nucleic acid probe can be separately contacted with the sample, either simultaneously or sequentially in any order, and the nucleic acid complex can be formed in situ in the sample. In some embodiments, the nucleic acid complex can be pre-formed and then contacted with the sample.

FIG. 1D shows an example where the nucleic acid probe comprises the target-binding region, the coupling oligonucleotide comprises the photosensitizer, and the splint oligonucleotide comprises the detectable label. The splint oligonucleotide can comprise a first hybridization region that hybridizes to a first complementary hybridization region in the nucleic acid probe, and the coupling oligonucleotide comprises a second hybridization region that hybridizes to a second complementary hybridization region in the nucleic acid probe. In some embodiments, the first complementary hybridization region is 3′ to the second complementary hybridization region. In some embodiments, the first complementary hybridization region is 5′ to the second complementary hybridization region. In some embodiments, the first and second complementary hybridization regions do not overlap. In some embodiments, the first and second complementary hybridization regions are directly linked by a phosphodiester bond. In some embodiments, the first and second complementary hybridization regions are linked by one or more nucleic acid residues. In some embodiments, hybridization of the splint oligonucleotide and the coupling oligonucleotide to the nucleic acid probe brings the detectable label and the photosensitizer in physical proximity, such that activation of the photosensitizer can attenuate the signal from the detectable label (e.g., via oxidizing the detectable label) while minimizing damage or crosslinking of other nearby molecules in the sample.

FIG. 1E shows an example where the nucleic acid probe comprises the target-binding region, one of multiple coupling oligonucleotides comprises the photosensitizer, and the splint oligonucleotide comprises the detectable label. In some embodiments, hybridization of the splint oligonucleotide and the coupling oligonucleotide comprising the photosensitizer brings the detectable label and the photosensitizer in physical proximity, such that activation of the photosensitizer can attenuate the signal from the detectable label (e.g., via oxidizing the detectable label) while minimizing damage or crosslinking of other nearby molecules in the sample.

Each of the multiple coupling oligonucleotides, independent of each other, can be coupled to the splint oligonucleotide via sequence complementarity and/or a covalent linkage (e.g., a covalent bond or linker). For example, the splint oligonucleotide, any one or more of the multiple coupling oligonucleotides, and/or the nucleic acid probe may comprise one or more photoreactive nucleotides or analogs thereof (e.g., ^{CNV}K, ^{CNV}D, or 3-cyanovinylcarbazole phosphoramidite), which can be activated to crosslink one strand of a duplex to the other strand (crosslinking is indicated by “X” in FIG. 1E). The splint oligonucleotide and the coupling oligonucleotides in FIG. 1E can be pre-coupled, and the pre-coupled oligonucleotides and the nucleic acid probe can be separately contacted with the sample, either simultaneously or sequentially in either order, so that the nucleic acid complex can be formed in situ in the sample. In some embodiments, the splint oligonucleotide, any one or more of the multiple coupling oligonucleotides, and the nucleic acid probe can be separately contacted with the sample, either simultaneously or sequentially in any order. In some embodiments, the nucleic acid complex can be pre-formed and then contacted with the sample.

Independent of each other, the splint oligonucleotide, the nucleic acid probe, and/or each of the multiple coupling oligonucleotides can be coupled to one or more functional moieties via a covalent linkage (e.g., a covalent bond or linker). Exemplary functional moieties are described in Section II-A-(iv) and can include an affinity moiety or a binding partner thereof and/or a reflective moiety.

Upon hybridization to the splint oligonucleotide, any two adjacent coupling oligonucleotides can comprise complementary overhangs that form a duplex. In some embodiments, the coupling oligonucleotide adjacent to the nucleic acid probe may comprise an overhang that hybridizes to a complementary overhang of the nucleic acid probe to form a duplex. Any one or more of the duplexes may but do not need to comprise covalent crosslinks (e.g., generated via ^{CNV}K, ^{CNV}D, or 3-cyanovinylcarbazole phosphoramidite). The duplex(es) may help stabilize the nucleic acid complex or a portion thereof such that the splint oligonucleotide and the coupling oligonucleotide(s) and optionally the nucleic acid probe can be removed together from the sample by applying a force to any of the molecules.

In some embodiments, the hybridization between the splint oligonucleotide and the nucleic acid probe has a first melting temperature (Tₘ) and the hybridization between the nucleic acid probe and the target sequence has a second Tₘ. In some embodiments, the first and second melting temperatures are different. In some embodiments, the first Tₘ is higher than the second Tₘ, such that the nucleic acid complex can be removed as a whole. In some embodiments, the first Tₘ is about 1° C., about 2° C., about 4° C., about 6° C., about 8° C., about 10° C., about 12° C., about 14° C., about 16° C., or more higher than the second Tₘ. For example, the sample can be washed under a temperature lower than the first Tₘ but higher than the second Tₘ, such that the nucleic acid probe can be removed from the target nucleic acid while it remains hybridized to the splint oligonucleotide.

A. Nucleic Acid Complex

Provided herein is a nucleic acid complex comprising a detectable label, a photosensitizer, and a target-binding region hybridized to a target sequence in a target nucleic acid in a biological sample.

(i) Detectable Label

In some embodiments, the terms “label” and “detectable label” comprise a directly or indirectly detectable moiety that is associated with (e.g., conjugated to) a molecule to be detected, e.g., a nucleic acid molecule of the nucleic acid complex that comprises a detectable label. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes (radioactive isotopes), fluorophores, fluorescers, chemiluminescent compounds, bioluminescent compounds, dyes, enzymes, enzyme substrates, enzyme cofactors, enzyme inhibitors, chromophores, dyes, metal ions, metal sols, ligands (e.g., biotin or haptens) and the like.

In some aspects, the detectable label comprises a luminophore. In some embodiments, the luminophore is a fluorophore. The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes (AlexaFluors), fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/P0-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

In some embodiments, the detectable label comprises an infrared fluorophore. An “infrared fluorophore” emits infrared light. In some embodiments, the infrared fluorophore has a longer excitation wavelength than a traditional fluorophore. An infrared fluorophore may be used as a detectable label in embodiments wherein the photosensitizer excites in the visible spectrum.

Examples of detectable labels comprise, but are not limited to, various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides (e.g., a nucleic acid molecule of the nucleic acid complex) conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), all of which are herein incorporated by reference in their entireties. In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label can comprise a signaling moiety that conveys information through the fluorescent absorption and/or emission properties of one or more molecules. Exemplary fluorescent properties comprise fluorescence intensity, fluorescence lifetime, emission spectrum characteristics and energy transfer.

In some embodiments, one or more detectable labels can be attached to a nucleic acid complex disclosed herein. In some embodiments, one or more detectable labels can be attached to a splint oligonucleotide of the nucleic acid complex. In some embodiments, one or more detectable labels can be attached to a nucleic acid probe of the nucleic acid complex. In some embodiments, one or more detectable labels can be attached to a coupling oligonucleotide of the nucleic acid complex. The one or more detectable labels can be incorporated during nucleic acid polymerization or amplification (e.g., Cy5®-labeled nucleotides, such as Cy5®-dCTP). Examples of commercially available fluorescent nucleotide analogues readily incorporated into nucleotide and/or polynucleotide sequences (e.g., a nucleic acid molecule of the nucleic acid complex) comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Any suitable method can be used for custom synthesis of nucleotides having other fluorophores (See, Henegariu et al. (2000) Nature Biotechnol. 18:345, the content of which is herein incorporated by reference in its entirety).

In some embodiments, one or more detectable labels can be attached to one or more molecules of a nucleic acid complex disclosed herein via post-synthetic attachment. In some embodiments, one or more detectable labels can be attached to a 3′ end of a splint oligonucleotide of the nucleic acid complex. In some embodiments, one or more detectable labels can be attached to a 3′ end of a nucleic acid probe of the nucleic acid complex. In some embodiments, one or more detectable labels can be attached to a 3′ end of a coupling oligonucleotide of the nucleic acid complex. In some embodiments, one or more detectable labels can be attached to a 5′ end of a splint oligonucleotide of the nucleic acid complex. In some embodiments, one or more detectable labels can be attached to a 5′ end of a nucleic acid probe of the nucleic acid complex. In some embodiments, one or more detectable labels can be attached to a 5′ end of a coupling oligonucleotide of the nucleic acid complex. In some embodiments, one or more detectable labels can be attached to a 3′ end of a splint oligonucleotide and one or more photosensitizers can be attached to a 5′ end of a coupling oligonucleotide. In some embodiments, one or more detectable labels can be attached to a 5′ end of a splint oligonucleotide and one or more photosensitizers can be attached to a 3′ end of a coupling oligonucleotide. In some embodiments, hybridization between the splint oligonucleotide and the coupling oligonucleotide brings the one or more detectable labels and the one or more photosensitizers into proximity, such that photosensitizer activation attenuates signals of the detectable label(s).

Fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62, the content of which is herein incorporated by reference in its entirety). In some embodiments, the metal particle is connected to an oligonucleotide (e.g., a coupling oligonucleotide or a splint oligonucleotide) by an oxidizable linker, which is cleaved when the ROS generated by the activation of the photosensitizer reacts with the linker for cleavage.

Biotin, or a derivative thereof, may also be used as a label on a nucleic acid molecule of the nucleic acid complex, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, an antibody can comprise an antibody molecule of any class, or any sub-fragment thereof, such as an Fab.

Other suitable labels for use in the methods provided herein may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleic acid molecule of the nucleic acid complex can be indirectly labeled, especially with a hapten that is then bound by a capture agent, e.g., as disclosed in U.S. Pat. Nos. 5,344,757, 5,702,888, 5,354,657, 5,198,537 and 4,849,336, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

In some embodiments, any of the detectable labels disclosed herein (e.g., a fluorescent label or a non-fluorescent label) can be connected to an oligonucleotide (e.g., a coupling oligonucleotide or a splint oligonucleotide) by an oxidizable linker, which is cleaved when the ROS generated by the activation of the photosensitizer reacts with the linker.

In some embodiments, the detectable label is detected in situ. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference). Further description of the detection of the detectable label can be found in Section IIB, below.

(ii) Photosensitizer

In some embodiments, the detectable label is in proximity with the photosensitizer in the nucleic acid complex. Photosensitizers are molecules (e.g., chemical compounds) which absorb light and become excited, then transfer the energy from the absorbed light into another nearby molecule. The absorbed light is often within the visible spectrum or infrared spectrum, as any higher energy electromagnetic radiation may result in the emission of elections (e.g., the photoelectric effect).

The photosensitizers of the present invention can be organometallic photosensitizers, organic photosensitizers, or nanomaterial photosensitizers. In some embodiments, the photosensitizer comprises a metal atom, such as iridum, ruthenium, or rhodium, fused to an organic ligand. In some embodiments, the photosensitizer comprises aromatic hydrocarbons. In some embodiments, the photosensitizer comprises quantum dots and/or nanorods. Exemplary photosensitizers that may be useful in the methods provided herein include, but are not limited to, naturally occurring porphyrins and their derivatives (e.g., Chlorophyll A or Chlorophyll B), tetrapyrroles, hemtoporphyrin, 5′-aminolevulinic acid, chlorins, bacteriochlorin, protoporphyrin IX, phthalocyanines, chlorins, benzoporphyrins, dyes (e.g., Toluidine Blue, Methylene Blue, or Rose Bengal), monoterpene, xanthene, and furocoymarians. In some embodiments, the photosensitizer comprises phthalocyanine or a derivative thereof, Atto-Thio12 (N-(9-(2-((3-carboxypropyl)(methyl)carbamoyl)phenyl)-6-(dimethylamino)-3H-thioxanthen-3-ylidene)-N-methylmethanaminium perchlorate), an organometallic photosensitizer, Methylene Blue (tetramethylthionine chloride or 3,7-bis(Dimethylamino)phenazathionium chloride), or Rose Bengal (4,5,6,7-Tetrachloro-2′,4′,5′,7′-tetraiodofluorescein disodium salt). In some embodiments, the organometallic photosensitizer is tris(2-phenylpyridine)iridium.

In some embodiments, the photosensitizer is activated in situ. Further description of the activation of the photosensitizer can be found in Section II.C, below.

(iii) Nucleic Acid Molecules of the Nucleic Acid Complex

The nucleic acid complex may comprise, in some embodiments, one, two, or more nucleic acid molecules. In some embodiments, the nucleic acid complex comprises one nucleic acid molecule comprising a detectable label, a photosensitizer, and a target-binding region hybridized to a target sequence in a target nucleic acid in a biological sample. In some embodiments, the nucleic acid molecule is a nucleic acid probe.

In some embodiments, the nucleic acid complex comprises two nucleic acid molecules. In some embodiments, the nucleic acid complex comprises a nucleic acid probe and a splint oligonucleotide hybridized thereto. In some embodiments, the complete sequence of the splint oligonucleotide is hybridized to the complete sequence of the entire probe (e.g., the sequence of the splint oligonucleotide is fully complementary to that of the nucleic acid probe). In some embodiments, the complete sequence of the splint oligonucleotide is hybridized to a complementary hybridization region in the nucleic acid probe. In some embodiments, the splint oligonucleotide comprises a hybridization region that hybridizes to a complementary hybridization region in the nucleic acid probe. In some embodiments, the hybridization region is a portion of the splint oligonucleotide. In some embodiments, the complementary hybridization region is a portion of the nucleic acid probe. In some embodiments, the nucleic acid probe comprises the target-binding region. In some embodiments, the target-binding region is the complete sequence of the nucleic acid probe. In some embodiments, the target-binding region is a portion of the complete sequence of the nucleic acid probe. In some embodiments, the nucleic acid probe and the splint oligonucleotide are independently about 10, 20, 30, 40, 50, 60, 70, 80, 90 or 100 or more nucleotides in length, or any integer (or range of integers) of nucleotides in between the indicated values.

In some embodiments, the nucleic acid probe comprises the photosensitizer. In some embodiments, the nucleic acid probe comprises the target-binding region and the photosensitizer. In some embodiments, the splint oligonucleotide comprises the detectable label. In some embodiments, the nucleic acid probe comprises the target-binding region and the photosensitizer, and the splint oligonucleotide comprises the detectable label. In some embodiments, the nucleic acid probe comprises the detectable label. In some embodiments, the nucleic acid probe comprises the target-binding region and the detectable label. In some embodiments, the splint oligonucleotide comprises the photosensitizer. In some embodiments, the nucleic acid probe comprises the target-binding region and the detectable label, and the splint oligonucleotide comprises the photosensitizer.

In some embodiments, the nucleic acid complex comprises a coupling oligonucleotide. In some embodiments, the coupling oligonucleotide is about 10, 20, 30, 40, 50, 60, 70, 80, 90 or 100 or more nucleotides in length, or any integer (or range of integers) of nucleotides in between the indicated values. In some embodiments, the coupling oligonucleotide hybridizes to the splint oligonucleotide. In some embodiments, the complete sequence of the coupling oligonucleotide hybridizes to the complete sequence of the splint oligonucleotide. In some embodiments, the complete sequence of the coupling oligonucleotide hybridizes to a portion of the complete sequence of the splint oligonucleotide. In some embodiments, a portion of the complete sequence of the coupling oligonucleotide hybridizes to a portion of the complete sequence of the splint oligonucleotide. In some embodiments, the coupling oligonucleotide hybridizes to the nucleic acid probe. In some embodiments, the complete sequence of the coupling oligonucleotide hybridizes to the complete sequence of the nucleic acid probe. In some embodiments, the complete sequence of the coupling oligonucleotide hybridizes to a portion of the complete sequence of the nucleic acid probe. In some embodiments, a portion of the complete sequence of the coupling oligonucleotide hybridizes to a portion of the complete sequence of the nucleic acid probe. In some embodiments, the coupling oligonucleotide hybridizes to the splint oligonucleotide and the nucleic acid probe. In some embodiments, the nucleic acid complex comprises two or more (such as two, three, four, five, or more) coupling oligonucleotides hybridized to the splint oligonucleotide. In some embodiments, the nucleic acid complex comprises two or more (such as two, three, four, five, or more) coupling oligonucleotides hybridized to the nucleic acid probe. In some embodiments, the nucleic acid complex comprises two or more (such as two, three, four, five, or more) coupling oligonucleotides hybridized to the splint oligonucleotide and the nucleic acid probe. In some embodiments, there is a gap, such as a gap of at least about one nucleotide (e.g., at least about two, three, four, or more, nucleotides), between the two or more coupling oligonucleotides hybridized to the splint oligonucleotide and/or the nucleic acid probe. In some embodiments, there is no gap between the two or more coupling oligonucleotides hybridized to the splint oligonucleotide and/or the nucleic acid probe.

In some embodiments, the splint oligonucleotide comprises: i) a hybridization region that hybridizes to a complementary hybridization region in the coupling oligonucleotide, and ii) a hybridization region that hybridizes to a complementary hybridization region in the nucleic acid probe. In some embodiments, the hybridization region that hybridizes to the complementary hybridization region in the coupling oligonucleotide is different in length from the hybridization region that hybridizes to the complementary hybridization region in the nucleic acid probe. In some embodiments, the hybridization region that hybridizes to the complementary hybridization region in the coupling oligonucleotide is different in sequence from the hybridization region that hybridizes to the complementary hybridization region in the nucleic acid probe. In some embodiments, the splint oligonucleotide comprises the detectable label. In some embodiments, the coupling oligonucleotide comprises the photosensitizer. In some embodiments, the splint oligonucleotide comprises the detectable label and the coupling oligonucleotide comprises the photosensitizer. In some embodiments, splint oligonucleotide comprises the photosensitizer. In some embodiments, the coupling oligonucleotide comprises the detectable label. In some embodiments, splint oligonucleotide comprises the photosensitizer and the coupling oligonucleotide comprises the detectable label. In some embodiments, the nucleic acid probe does not comprise the photosensitizer. In some embodiments, the nucleic acid probe does not comprise the detectable label. In some embodiments, the nucleic acid probe comprises neither the photosensitizer or the detectable label.

In some embodiments, the splint oligonucleotide and the coupling oligonucleotide are configured to covalently couple to each other. In some embodiments, the splint oligonucleotide and the coupling oligonucleotide are configured to noncovalently couple to each other. In some embodiments, the splint oligonucleotide and the coupling oligonucleotide are configured to covalently and noncovalently couple to each other. For example, any technique that increases the binding strength between the coupling oligonucleotide and the splint oligonucleotide or crosslinks the coupling oligonucleotide and the splint oligonucleotide may be used to couple the two oligonucleotides. In some embodiments, the splint oligonucleotide comprises one or more photoreactive nucleotides capable of reacting with a nucleotide in an oligonucleotide strand hybridized thereto (e.g., a coupling oligonucleotide and/or a nucleic acid probe) to form a covalent bond. In some embodiments, the covalent bond is formed by click-chemistry.

In some embodiments, the coupling oligonucleotide comprises one or more photoreactive nucleotides capable of reacting with a nucleotide in an oligonucleotide strand hybridized thereto (e.g., a splint oligonucleotide and/or a nucleic acid probe) to form a covalent bond. In some embodiments, the splint oligonucleotide and the coupling oligonucleotide each independently comprises a photoreactive nucleotide capable of reacting with a nucleotide in an oligonucleotide strand hybridized thereto to form a covalent bond. In some embodiments, the photoreactive nucleotide comprises a 3-cyanovinylcarbazole (^{CNV}K) nucleoside, 3-cyanovinylcarbazole modified D-threoninol (^{CNV}D), a 3-cyanovinylcarbazole phosphoramidite, or a psoralen. Psoralens are molecules that can intercalate with nucleic acid (e.g., DNA) and that upon irradiation can form covalent bonds with pyrimidines (C/T/U). In the absence of irradiation, psoralens bind non-covalently similarly to any other intercalating agent, and covalent crosslinking to nucleic acid depends on irradiation. In some embodiments, psoralen-modified nucleotides can be incorporated into oligonucleotides for site-specific photo-crosslinking. In some embodiments, the photoreactive nucleotide can comprise a universal base (e.g., 2′-deoxyinosine and 2′-deoxynebularine). In some embodiments, the method further comprises photo-activating the photoreactive nucleotide to covalently couple the splint oligonucleotide and the coupling oligonucleotide. In some embodiments, the photo-activating is performed using a light having a wavelength of about 350 nm to about 400 nm. In some embodiments, the photo-activating is performed using a light having a wavelength greater than about 350 nm, such as greater than any of about 360 nm, 370 nm, 380 nm, 390 nm, 400 nm, or greater. In some embodiments, the photo-activating is performed using a light having a wavelength less than about 400 nm, such as less than any of about 390 nm, 380 nm, 370 nm, 360 nm, 350 nm, or less. In some embodiments, the photo-activating is performed prior to the splint oligonucleotide contacting the biological sample. In some embodiments, the photo-activating is performed prior to the coupling oligonucleotide contacting the biological sample. In some embodiments, the photo-activating is performed prior to the splint oligonucleotide and the coupling oligonucleotide contacting the biological sample. In some embodiments, the photo-activating is performed without activating the photosensitizer.

In some embodiments, the splint oligonucleotide comprises the detectable label, and the method comprises: contacting the splint oligonucleotide and the coupling oligonucleotide with an oligonucleotide comprising the photosensitizer; ligating the oligonucleotide comprising the photosensitizer to the coupling oligonucleotide using the splint oligonucleotide as a template, whereby the photosensitizer and the detectable label are brought into proximity with each other. In some embodiments, the splint oligonucleotide and the coupling oligonucleotide are covalently coupled to each other prior to or after the ligation. In some embodiments, the splint oligonucleotide and the coupling oligonucleotide are covalently coupled to each other using any of the methods provided herein.

In some embodiments, the nucleic acid complex is formed prior to contacting with the biological sample. In some embodiments, any two of the nucleic acid probe, the splint oligonucleotide, and the coupling oligonucleotide, form a complex prior to contacting with the biological sample. For example, in some embodiments, the splint oligonucleotide and the coupling oligonucleotide form a complex prior to contacting with the biological sample. In some embodiments, the splint oligonucleotide and the nucleic acid probe form a complex prior to contacting with the biological sample. In some embodiments, the nucleic acid probe and the splint oligonucleotide form a complex in the biological sample prior to the coupling oligonucleotide contacting the biological sample. In some embodiments, the coupling oligonucleotide and the nucleic acid probe form a complex prior to contacting with the biological sample. In some embodiments, each of the nucleic acid probe, the splint oligonucleotide, and the coupling oligonucleotide form a complex prior to contacting with the biological sample.

In some embodiments, the splint oligonucleotide and the coupling oligonucleotide form a complex outside the biological sample prior to the complex contacting the biological sample. In some embodiments, the nucleic acid probe and the complex (e.g., the complex comprising the splint oligonucleotide and the coupling oligonucleotide) are contacted with the biological sample simultaneously. In some embodiments, the nucleic acid probe and the complex are pre-mixed, and are contacted with the biological sample simultaneously. In some embodiments, the nucleic acid probe and the complex are in separate reagent mixtures, wherein each regent mixture is contacted with the biological sample simultaneously. In some embodiments, the nucleic acid probe and the complex are contacted with the biological sample sequentially. For example, the nucleic acid probe may be contacted with the biological sample prior to the complex. In other examples, the complex may be contacted with the biological sample prior to the nucleic acid probe. In some embodiments, the nucleic acid probe is hybridized to the target nucleic acid in the biological sample prior to the complex contacting the biological sample. In some embodiments, the nucleic acid probe, the splint oligonucleotide, and the coupling oligonucleotide form a complex outside the biological sample prior to the complex contacting the biological sample.

(iv) Optional Functional Moieties of the Nucleic Acid Complex

The nucleic acid complex may comprise additional functional moieties that can further improve in situ analysis. For example, functional moieties such as affinity moieties can be incorporated into the nucleic acid complex in order to physically separate the nucleic acid complex from the biological sample following detection of a signal associated with a detectable label. In other examples, reflective moieties are incorporated into the nucleic acid complex.

In some embodiments, the nucleic acid complex further comprises an affinity moiety. In some embodiments, an affinity moiety comprises a chemical group or molecule that can bind a partner such as a receptor or protein. Examples of affinity moieties include, but are not limited to, biotin, des-biotin and other derivatives, dinitrophenol, dansyl, fluorescein, Cy5, and digoxigenin, avidin, and streptavidin. In some embodiments, the affinity moiety is covalently coupled to the nucleic acid complex. In some embodiments, the affinity moiety is covalently coupled to the nucleic acid complex via a covalent bond. In some embodiments, the affinity moiety is covalently coupled to the nucleic acid complex via a linker. Any of the nucleic acid molecules of a nucleic acid complex described herein may comprise one or more affinity moieties. In some embodiments, a probe or oligonucleotide, such as any of the molecules of a nucleic acid complex described herein, comprises one, two, or more affinity moieties. In some embodiments, a splint oligonucleotide, such as any of the splint oligonucleotides of a nucleic acid complex described herein, comprises one, two, or more affinity moieties. In some embodiments, a coupling oligonucleotide, such as any of the coupling oligonucleotides of a nucleic acid complex described herein, comprises one, two, or more affinity moieties. In some embodiments, a nucleic acid probe and a splint oligonucleotide can each comprise one, two, or more affinity moieties. In some embodiments, a nucleic acid probe and a coupling oligonucleotide can each comprise one, two, or more affinity moieties. In some embodiments, a splint oligonucleotide and a coupling oligonucleotide can each comprise one, two, or more affinity moieties. In some embodiments, a nucleic acid probe, a splint oligonucleotide, and a coupling oligonucleotide herein can each comprise one, two, or more affinity moieties. In some embodiments, the affinity moiety comprises a biotin or derivative thereof.

In some embodiments, the method comprises contacting the biological sample with a binding partner of the affinity moiety. In some embodiments, a binding partner comprises a chemical group or molecule, such as a protein or a receptor, that specifically binds to the affinity moiety. In some embodiments, the biological sample is contacted with the binding partner after the signal associated with the detectable label is detected. In some embodiments, the binding partner is coupled to a nanoparticle. A nanoparticle may be generated, for example, by spark ablation. In some embodiments, the binding partner is coupled to a diamagnetic particle. In some embodiments, the binding partner is coupled to a paramagnetic particle. In some embodiments, the binding partner is coupled to a ferromagnetic particle.

In some embodiments, the method further comprises removing a complex comprising the binding partner (e.g., the binding partner that is bound to the affinity moiety) and the nucleic acid probe, the splint oligonucleotide, and/or the coupling oligonucleotide from the biological sample. In some embodiments, one or more molecules in a nucleic acid complex disclosed herein can comprise a flexible linker with an affinity moiety to facilitate subsequent physical removal of the nucleic acid complex or a portion thereof. For example, a nucleic acid probe, splint oligonucleotide, and/or coupling oligonucleotide of the nucleic acid complex may comprise a biotin or derivative thereof. The biotin or derivative thereof can be attached to the 3′ or 5′ end of a nucleic acid probe, a coupling oligonucleotide, and/or a splint oligonucleotide of the nucleic acid complex, for subsequent magnetic bead capture and removal. Following the detection of the signal associated with the detectable label, the biological sample may be contacted with streptavidin (e.g., streptavidin particles that are binding partners of the biotin affinity moiety). The streptavidin can bind the biotin of a nucleic acid molecule (e.g., a nucleic acid probe, splint oligonucleotide, and/or coupling oligonucleotide) of the nucleic acid complex, and the method may comprise removing the streptavidin and the nucleic acid molecule (e.g., a nucleic acid probe, splint oligonucleotide, and/or coupling oligonucleotide) of the nucleic acid complex comprising the biotin from the biological sample.

In some embodiments, the nucleic acid complex comprises a reflective moiety. In some embodiments, the reflective moiety comprises a chemical group or molecule that can absorb and scatter light. Exemplary reflective moieties include, but are not limited to gold nanoparticles, gadolinium, carbon dots, and silver nanoparticles. In some embodiments, the reflective moiety is covalently coupled to the nucleic acid complex. In some embodiments, the reflective moiety is covalently coupled to the nucleic acid complex via a covalent bond. In some embodiments, the reflective moiety is covalently coupled to the nucleic acid complex via a linker. Any of the nucleic acid molecules of a nucleic acid complex described herein may comprise one or more reflective moieties. In some embodiments, a nucleic acid probe, such as any of the nucleic acid probes of a nucleic acid complex described herein, comprises one, two, or more reflective moieties. In some embodiments, a splint oligonucleotide, such as any of the splint oligonucleotides of a nucleic acid complex described herein, comprises one, two, or more reflective moieties. In some embodiments, a coupling oligonucleotide, such as any of the coupling oligonucleotides of a nucleic acid complex described herein, comprises one, two, or more reflective moieties. In some embodiments, a nucleic acid probe and a splint oligonucleotide can each comprise one, two, or more reflective moieties. In some embodiments, a nucleic acid probe and a coupling oligonucleotide can each comprise one, two, or more reflective moieties. In some embodiments, a splint oligonucleotide and a coupling oligonucleotide can each comprise one, two, or more reflective moieties. In some embodiments, a nucleic acid probe, a splint oligonucleotide, and a coupling oligonucleotide can each comprise one, two, or more reflective moieties. In some embodiments, the reflective moiety reflects infrared light. In some embodiments, the reflective moiety comprises a gold nanoparticle.

(v) Target-Binding Region and Target Nucleic Acids

The nucleic acid complex comprises a target-binding region hybridized to a target sequence within a target nucleic acid of the biological sample. Various types of target nucleic acids and target sequences thereof are contemplated herein that are compatible with the provided methods. Such target sequences and analytes (e.g., endogenous analytes, labelling agents, and products of endogenous analytes) are described, and in Section IIIB. In some embodiments, the target nucleic acid comprises a cellular nucleic acid molecule. In some embodiments, the target nucleic acid is a cellular nucleic acid molecule. In some embodiments, the target nucleic acid comprises a product of a cellular nucleic acid molecule (e.g., an amplification product, such as a rolling circle amplification (RCA) product (RCP). In some embodiments, the target nucleic acid is a product of a cellular nucleic acid molecule. In some embodiments, the cellular nucleic acid molecule or product thereof is a genomic DNA, mRNA, or cDNA.

In some embodiments, the target nucleic acid comprises a primary probe that hybridizes to a cellular nucleic acid molecule or a product thereof. In some embodiments, the target nucleic acid is a primary probe that hybridizes to a cellular nucleic acid molecule or a product thereof. In some embodiments, the primary probe is a primary probe comprising a 3′ or 5′ overhang upon hybridization to the cellular nucleic acid molecule or product thereof (e.g., intermediate probe such as an L-probe). In some embodiments, the primary probe is a primary probe comprising a 3′ overhang and a 5′ overhang upon hybridization to the cellular nucleic acid molecule or product thereof (e.g., U-probe). In some embodiments, the primary probe is a circular primary probe. In some embodiments, the primary probe is a circularizable primary probe or probe set (e.g., padlock probe, split FISH probe, SNAIL probe, etc.). In some embodiments, the primary probe is a primary probe or probe set comprising a split hybridization region configured to hybridize to a splint (e.g., Z-probe/RNAscope, upside down U probe, SNAIL probe, PLAYR probe, PLISH probe, etc.). In some embodiments, the primary probe is selected from the group consisting of: a primary probe comprising a 3′ or 5′ overhang upon hybridization to the cellular nucleic acid molecule or product thereof, a primary probe comprising a 3′ overhang and a 5′ overhang upon hybridization to the cellular nucleic acid molecule or product thereof; a circular primary probe; a circularizable primary probe or probe set; a primary probe or probe set comprising a split hybridization region configured to hybridize to a splint; and a combination thereof.

In some embodiments, the 3′ overhang and the 5′ overhang of the primary probe each independently comprises one or more barcode sequences. In some embodiments, the target-binding region hybridizes to the one or more barcode sequences in the primary probe. In some embodiments, the one or more barcode sequences comprise a split barcode sequence.

In some embodiments, the target nucleic acid comprises or is an intermediate probe that hybridizes to a primary probe or a product or complex thereof, wherein the primary probe hybridizes to a cellular nucleic acid molecule or a product thereof. In some embodiments, the product of the primary probe is a RCP. In some embodiments, the complex of the primary probe is a complex comprising an initiator and an amplifier for hybridization chain reaction (HCR). HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401), all of which are incorporated herein by reference. In some embodiments, the complex of the primary probe is a complex comprising an initiator and an amplifier for linear oligonucleotide hybridization chain reaction (LO-HCR). Exemplary methods and compositions for LO-HCR are described in US 2021/0198723, incorporated herein by reference in its entirety. In some embodiments, the product of the primary probe is a primer exchange reaction (PER) product. See e.g., US 2019/0106733, which is incorporated herein by reference, for exemplary molecules and PER reaction components. In some embodiments, the complex of the primary probe is a complex comprising a pre-amplifier and an amplifier for branched DNA (bDNA). For exemplary complexes, see e.g., US 2020/0399689 and US 2022/0064697, which are fully incorporated by reference herein. In some embodiments, a detection reagent used in HCR, LO-HCR, PER, or bDNA-based methods can comprise a photosensitizer or a sequence for hybridization of an oligonucleotide comprising a photosensitizer, such that the detection reagent comprises a detectable label in proximity with a photosensitizer. For instance, the target sequence as shown in FIGS. 1A-1E can be in a detection reagent used in HCR, LO-HCR, PER, or bDNA-based methods. The target sequence can be in a complex formed in in HCR, LO-HCR, PER, or bDNA-based methods. For example, the target sequence as shown in FIGS. 1A-1E can be in an HCR initiator or monomer, a LO-HCR initiator or monomer, an oligonucleotide in PER, or a pre-amplifier or amplifier in bDNA-based method.

In some embodiments, the intermediate probe is an intermediate probe comprising an overhang upon hybridization to the primary probe or product or complex thereof. In some embodiments, the intermediate probe is comprising a 3′ or 5′ overhang upon hybridization to the primary probe or product or complex thereof (e.g., an L-probe). In some embodiments, the intermediate probe is an intermediate probe comprising a 3′ overhang and a 5′ overhang upon hybridization to the primary probe or product or complex thereof (e.g., a U-probe). In some embodiments, the intermediate probe is a circular intermediate probe. In some embodiments, the intermediate probe is a circularizable intermediate probe or probe set. In some embodiments, the intermediate probe is an intermediate probe or probe set comprising a split hybridization region configured to hybridize to a splint. In some embodiments, the intermediate probe is selected from the group consisting of: an intermediate probe comprising a 3′ or 5′ overhang upon hybridization to the primary probe or product or complex thereof, an intermediate probe comprising a 3′ overhang and a 5′ overhang upon hybridization to the primary probe or product or complex thereof, a circular intermediate probe; a circularizable intermediate probe or probe set; an intermediate probe or probe set comprising a split hybridization region configured to hybridize to a splint; and a combination thereof. In some embodiments, the intermediate probe hybridizes to one or more barcode sequences in the primary probe or product or complex thereof.

In some embodiments, the intermediate probe (e.g., an L-probe or a U-probe) hybridizes to a target sequence (e.g., a target sequence in a target nucleic acid) in the biological sample. In some embodiments, the intermediate probe hybridizes to the primary probe or product or complex thereof, a splint, a barcode sequence in the primary probe or product or complex thereof, or a combination thereof. In some embodiments, the intermediate probe comprises a region that hybridizes to the target sequence. In some embodiments, the intermediate probe comprises a first region that hybridizes to the target sequence and a second region that hybridizes to the the primary probe or product or complex thereof, a splint, a barcode sequence in the primary probe or product or complex thereof, or a combination thereof.

In some embodiments, the target nucleic acid comprises or is a reporter oligonucleotide of a labelling agent. In some embodiments, the labelling agent comprises an analyte-binding region and the reporter oligonucleotide. In some embodiments, the analyte comprises a nucleic acid, a protein, a carbohydrate, a lipid, or a small molecule, or a complex thereof.

In some embodiments, the target nucleic acid comprises an overhang region comprising multiple copies of the target sequence. In some embodiments, the target nucleic acid is concatemeric (e.g., a nucleic acid concatemer) and comprises multiple copies of the target sequence. In some embodiments, the target nucleic acid comprises a RCP of a circular or circularized probe that hybridizes to a nucleic acid molecule in the biological sample. In some embodiments, the circular or circularized probe comprises a barcode region.

In some embodiments, the method further comprises generating the target nucleic acid or a molecule or complex to which the target nucleic acid directly or indirectly binds. In some embodiments, the target nucleic acid or the molecule or complex is generated in situ in the biological sample. In some embodiments, generating the target nucleic acid or the molecule or complex comprises: (i) contacting the biological sample with a circular or circularizable probe or probe set that hybridizes to a nucleic acid molecule in the biological sample; and (ii) performing a RCA using the circular probe or a circularized probe as template. In some embodiments, the circularized probe is generated by circularizing the circularizable probe or probe set, such as by any of the methods described herein.

B. Detecting a Signal

In some aspects, the provided methods involve analyzing, e.g., detecting or determining, one or more sequences (e.g., target sequences) hybridized to the nucleic acid complexes described herein. In some embodiments, the detecting comprises detecting the detectable label of the nucleic acid complex, or via hybridization to adaptor probes that hybridize to the nucleic acid complex. In some embodiments, the analysis comprises determining the sequence of all or a portion of the target sequence (e.g., a barcode sequence or a complement thereof), wherein the sequence is indicative of a sequence of the target nucleic acid in the biological sample.

In some cases, analysis is performed on one or more images captured, and may comprise processing the image(s) and/or quantifying signals observed. In some embodiments, images of signals from different fluorescent channels and/or nucleic acid complex hybridization cycles can be compared and analyzed. In some embodiments, images of signals (or absence thereof) at a particular location in a sample from different fluorescent channels and/or sequential nucleic acid complex hybridization cycles can be aligned to analyze an analyte at the location. For instance, a particular location in a sample can be tracked and signal spots from sequential nucleic acid complex (and optionally ligation) cycles can be analyzed to detect a target polynucleotide sequence (e.g., a barcode sequence or subsequence thereof) in a target nucleic acid at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some cases, the analysis further includes displaying the information from the analysis or detection step. In some embodiments, software may be used to automate the processing, analysis, and/or display of data.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization in a FISH-type assay).

In some embodiments, the methods comprise detecting the sequence of all or a portion of a target nucleic acid or a product thereof, or a primary probe or a product thereof, such as one or more barcode sequences present in the primary probe. In some embodiments, the sequence of the primary probe, or barcode thereof, is indicative of a sequence of the target nucleic acid to which the nucleic acid complex is hybridized (e.g., via a target-binding region hybridized to a target sequence of the target nucleic acid. In some embodiments, the analysis and/or sequence determination comprises detecting the sequence of all or a portion of the target nucleic acid or a product thereof, or the primary probe or a product thereof and/or in situ hybridization to the target nucleic acid or a product thereof, or the primary probe or a product thereof. In some embodiments, detecting the sequence involves sequencing by hybridization, sequencing by ligation, sequencing by synthesis, sequencing by binding, and/or fluorescent in situ sequencing (FISSEQ), hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US2017/0009278, which is incorporated herein by reference, for exemplary probes and HCR reaction components. In some embodiments, the detection or determination comprises hybridizing to the first overhang a detection oligonucleotide labeled with a fluorophore, an isotope, a mass tag, or a combination thereof. In some embodiments, the detection or determination comprises imaging one or more detectably labeled probes hybridized to the target nucleic acid. In some embodiments, the target nucleic acid is an mRNA in a tissue sample, and the detection or determination is performed when the target nucleic acid and/or the amplification product is at a location in situ in the tissue sample. In some embodiments, the target nucleic acid is an amplification product (e.g., a nucleic acid concatemer).

In some aspects, the provided methods comprise imaging the nucleic acid complex, for example, via detecting a signal associated with the detectable label of the nucleic acid complex. In some embodiments, the detectable label can be measured and quantitated. In some embodiments, the signal associated with the detectable label is detected without activating the photosensitizer in the nucleic acid complex. In some embodiments, the signal associated with the detectable label is before activating the photosensitizer in the nucleic acid complex. The signal associated with the detectable label can be any signal associated with any of the detectable labels described herein, and can be detected using any suitable method. In some embodiments, the signal associated with the detectable label may be a fluorescent signal (e.g., when the detectable label is a fluorophore)

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable label can be used to detect one or more nucleic acid complexes hybridized to a target sequence, described herein. In some embodiments, the methods involve incubating the nucleic acid complex containing the detectable label with the sample, washing unbound nucleic acid complex, and detecting the label, e.g., by imaging. In some embodiments, the nucleic acid complex remains hybridized to the target nucleic acid during the washing and detecting steps. In some embodiments, a primary probe, intermediate probe, or product(s) thereof remain crosslinked to the target nucleic acid during the washing and detecting steps.

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the signal associated with the detectable label. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the signal associated with the detectable label. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (e.g., reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequence detection can be performed by sequential fluorescence hybridization. Sequential fluorescence hybridization can involve sequential hybridization of detection probes comprising an oligonucleotide and a detectable label. In some aspects, nucleic acid complexes each comprising a detectable label, a photosensitizer, and a target-binding region for hybridizing to the target sequence is used as the detection probe.

In addition to the detection methods described herein, sequence detection can be additionally performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; US2020/0080139 A1; US20210017587 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequence detection. These methods utilize labeled nucleic acid decoder probes that are complementary to at least a portion of a barcode sequence. Provided herein are hybridization methods using nucleic acid complexes for the detection of target sequences. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), each of which are herein incorporated by reference in their entireties.

In addition to the detection methods described herein, real-time monitoring of DNA polymerase activity can be additionally performed. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, all of which are herein incorporated by reference in their entireties.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds. In some embodiments, the analysis and/or sequence determination involves washing to remove unbound polynucleotides, thereafter revealing a fluorescent product for imaging.

C. Activating the Photosensitizer

A method for analyzing a biological sample provided herein comprises activating the photosensitizer of the nucleic acid complex. Activating the photosensitizer attenuates the signal associated with the detectable label.

In some embodiments, the photosensitizer is a Type I photosensitizer activated by a light source into a triplet state. A triplet state is an electronic state in which two electrons in different molecular orbitals have parallel spin. The excited, triplet state photosensitizer may react with the detectable label (e.g., not molecular oxygen) of the nucleic acid complex to reform the relaxed, ground state photosensitizer, thereby attenuating the signal associated with the detectable label.

In some embodiments, the photosensitizer is a Type II photosensitizer activated by a light source into a triplet state. The excited photosensitizer may react with ground state, triplet oxygen molecule, thereby exciting the oxygen molecule into the singlet state (e.g., forming a reactive oxygen species (ROS)). The excited, singlet oxygen molecule can then react with the detectable label of the nucleic acid complex, thereby attenuating the signal associated with the detectable label.

In some embodiments, the photosensitizer is activated using an infrared light. In some embodiments, the photosensitizer is activated using a light having a wavelength between about 800 nm and about 1 mm, such as between about 800 nm and about 900 nm, between about 850 nm and about 950 nm, or between about 900 nm and about 1,000 nm. In some embodiments, the photosensitizer is activated using a light having a wavelength of less than about 1,000 nm, such as less than any of about 950 nm, 900 nm, 850 nm, 800 nm, or less. In some embodiments, the photosensitizer is activated using a light having a wavelength of greater than about 800 nm, such as greater than any of about 850 nm, 900 nm, 950 nm, 1,000 nm, or more. In some embodiments, the photosensitizer is activated using near infrared light. In some embodiments, the photosensitizer is activated using a light having a wavelength between about 800 nm and about 2,500 nm, such as between about 800 nm and about 1,500 nm, between about 1,000 nm and about 2,000 nm, or between about 1,500 nm and about 2,500 nm. In some embodiments, the photosensitizer is activated using a light having a wavelength of less than about 2,500 nm, such as less than any of about 2,000 nm, 1,500 nm, 1,000 nm, 950 nm, 900 nm, 850 nm, 800 nm, or less. In some embodiments, the photosensitizer is activated using a light having a wavelength of greater than about 800 nm, such as greater than any of about 850 nm, 900 nm, 950 nm, 1,000 nm, 1,500 nm, 2,000 nm, 2,500 nm, or more. In some embodiments, the photosensitizer is activated using visible light.

In some embodiments, activation of the photosensitizer leads to oxidation of the detectable label. In some embodiments, activation of the photosensitizer generates a ROS. A ROS is a reactive chemical formed from molecular oxygen (O₂). In some embodiments, the ROS comprises a free radical. In some embodiments, the ROS comprises a non-radical. In some embodiments, the ROS comprises a combination of a free radical and a non-radical. The ROS may be any suitable ROS. In some embodiments, the ROS comprises alpha-oxygen (α-O). In some embodiments, the ROS comprises superoxide (O^{•-}₂). In some embodiments, the ROS comprises a hydroxyl radical (OH^{•}). In some embodiments, the ROS comprises a hydrogen peroxide (H₂O₂). In some embodiments, the ROS comprises singlet oxygen (¹O₂). In some embodiments, the ROS comprises a combination of superoxide, hydroxyl radical, hydrogen peroxide, singlet oxygen, and/or alpha-oxygen. In some embodiments, the ROS reacts with the detectable label to render it undetectable under the conditions for the detecting the signal associated with the detectable label. In some embodiments, the ROS extinguishes the signal associated with the detectable label. In some embodiments, the ROS permanently extinguishes the signal associated with the detectable label.

In some embodiments, once generated (e.g., excited), the ROS has a lifetime of between about 1 nanosecond (ns) and about 40 ns, such as between about 1 ns and about 20 ns, between about 10 ns and about 30 ns, or between about 20 ns and about 40 ns. In some embodiments, the ROS has a lifetime of less than about 40 ns after excitation, such as less than any of about 35 ns, 30 ns, 25 ns, 20 ns, 15 ns, 10 ns, 5 ns, 1 ns, or less, after excitation. In some embodiments, the ROS has a lifetime of greater than about 1 ns after excitation, such as greater than any of about 5 ns, 10 ns, 15 ns, 20 ns, 25 ns, 30 ns, 35 ns, 40 ns, or greater.

The ROS, such as singlet oxygen, can become quenched (e.g., decays) as it diffuses away from the location at which it is generated (e.g., at the photosensitizer) within the biological sample. In some embodiments, the ROS has a diffusional radius between about 0.1 nm and about 50 nm from the photosensitizer, such as between about 0.1 nm and about 10 nm, between about 5 nm and about 20 nm, between about 10 nm and about 30 nm, or between about 20 nm and about 50 nm, from the photosensitizer. In some embodiments, the ROS has a diffusional radius of greater than about 0.1 nm from the photosensitizer, such as greater than any of about, 1 nm, 5 nm, 10 nm, 20 nm, 25 nm, 30 nm, 35 nm, 40 nm, 45 nm, 50 nm, or greater, from the photosensitizer. In some embodiments, the ROS has a diffusional radius of less than about 50 nm from the photosensitizer, such as less than about 45 nm, 40 nm, 35 nm, 30 nm, 25 nm, 20 nm, 10 nm, 5 nm, 1 nm, 0.1 nm, or less, from the photosensitizer. In some embodiments, the ROS decays to between about 1% and about 50%, such as between about 1% and about 20%, between about 10% and about 30%, between about 20% and about 40%, or between about 30% and about 50%. In some embodiments, the ROS decays to below about 50%, such as below any of about 40%, 30%, 20% 10%, 1%, or less. In some embodiments, the ROS decays greater than about 50%, such as greater than about 60%, 70%, 80%, 90%, 95%, 99%, or greater. In some embodiments, the ROS decays to below 50%, below 10%, or below 1% within 0.1 nm, 1 nm, 5 nm, 10 nm, 20 nm, 25 nm, 30 nm, 35 nm, 40 nm, 45 nm, or 50 nm from the photosensitizer.

D. Signal Detection and Attenuation in Sequential Cycles

In some embodiments, hybridization of a nucleic acid probe, a splint oligonucleotide, and optionally a coupling oligonucleotide, to form a nucleic acid complex in situ can be performed in sequential cycles.

FIG. 2 depicts in situ hybridization of the nucleic acid complex to a target nucleic acid with one or more target sequence that hybridizes to the nucleic acid probe of the nucleic acid complex. In some instances, the target nucleic acid may be a RCA-generated amplification product comprising multiple copies of a target sequence (left panel of FIG. 2). In such instances, a suitable primary probe or probe set (e.g., circular or circularizable probes hybridized to the cellular nucleic acid) can be used to generate an RCA product in one or more steps (as indicated by the block arrow in FIG. 2, left panel). In some instances, the target nucleic acid may be a primary probe comprising a region that is complementary to and hybridizes to the cellular nucleic acid and an overhang region comprising one or more target sequence(s) (middle panel of FIG. 2). In some instances, the target nucleic acid may be an intermediate probe comprising a region that is complementary to and hybridizes to a primary probe, the primary probe comprises a region that is complementary to and hybridizes to the cellular nucleic acid; the intermediate probe also comprises an overhang region comprising multiple copies of a target sequence (right panel of FIG. 2).

In some embodiments, in situ hybridization of a plurality of nucleic acid complexes in sequential cycles is used to decode the combinations of barcodes in the primary probes and detect the analytes that the primary probes bind to. For instance, each of Gene X, Gene Y, and Gene Z (e.g., RNA transcripts of the genes) can be targeted by a plurality of primary probes, each comprising a target-hybridizing region and a barcode region. The primary probes for Gene X comprise Barcodes I and II, the primary probes for Gene Y comprise Barcode I, and the primary probes for Gene Z comprise Barcodes II and III. In this example, Barcodes I-III are different, and the combinations of the barcodes in the primary probes can be used to encode the three genes. Signals associated with the barcodes at one or more locations in a sample can be detected in sequential hybridization cycles (using nucleic acid complexes disclosed herein as detection reagents) and used to decode the combinations of the barcodes, thereby decoding and detecting the three genes at the one or more locations in the sample. Nucleic acid complexes each comprising a target-binding region (binding to Barcode I, II, or III) and a detectable label/photosensitizer pair are used in sequential cycles to detect the signals associated with the barcodes in the primary probes.

A plurality of nucleic acid complexes can be generated to provide a library of nucleic acid complexes, each comprising a target-binding region for a different analyte. Multiple libraries of nucleic acid complexes can be generated for use to combinatorially detect a plurality of analytes at one or more locations in a biological sample. For instance, in FIG. 3, a first library of nucleic acid complexes (for Cycle 1) can be generated using nucleic acid probes each comprising i) a barcode for a particular analyte, e.g., a barcode for Gene X, Gene Y, and Gene Z, respectively, and ii) a complement of a detection oligonucleotide (DO) sequence. Each nucleic acid complex can comprise a coupling oligonucleotide comprising a photosensitizer coupled to a splint oligonucleotide, the splint oligonucleotide comprising the DO sequence that hybridizes to the nucleic acid probe in the nucleic acid complex. Similarly, a second library of nucleic acid complexes (for Cycle 2) and a third library of nucleic acid complexes (for Cycle 3) can be generated, each comprising a barcode for Gene X, Gene Y, or Gene Z. The same DO sequence may be associated with different gene-specific barcodes in the same library of nucleic acid complexes. Between nucleic acid complexes in different libraries, the same gene-specific barcode can be associated with different DO sequences or the same DO sequence.

Each library of nucleic acid complexes can be contacted with a biological sample in a hybridization cycle, and the libraries of nucleic acid complexes can be applied in sequential probe hybridization cycles. The nucleic acid complexes in each library can be pre-formed prior to contacting the sample, or each nucleic acid complex can be provided in two or more parts and the nucleic acid complex can be assembled in situ. For instance, a library of nucleic acid probes comprising complements of DO sequences and gene-specific barcodes can be contacted with the sample, followed by contacting the sample with a library of coupling oligonucleotides (each comprising a photosensitizer) coupled to splint oligonucleotides (each comprising a DO sequence and a detectable label corresponding to the DO sequence).

Each DO sequence can correspond to a different “color,” for example, DO1 to blue (“B”), DO2 to green (“G”), DO3 to yellow (“Y”), and DO4 to red (“R”). FIG. 3 shows that signals associated with the nucleic acid complexes (each of which may comprise a gene-specific barcode) at one or more locations in a sample can be detected in the sequential probe hybridization cycles, and the temporal combination or pattern of the “colors” can be detected and used to decode the gene-specific barcodes, thereby decoding and detecting the three genes at the one or more locations in the sample. The gene-specific barcode in each nucleic acid complex can bind to a sequence (e.g., a complementary barcode sequence) in an RCA product (e.g., as shown in FIG. 2, left panel), a sequence in a primary probe (e.g., as shown in FIG. 2, middle panel), or a sequence in an intermediate probe (e.g., as shown in FIG. 2, right panel) which may be in a branched hybridization complex.

In some aspects, the provided methods involve analyzing, e.g., detecting or determining, one or more nucleic acid sequences. In some cases, the analysis is performed on one or more images captured, and may comprise processing the image(s) and/or quantifying signals observed. In some embodiments, the analysis comprises detecting a sequence (e.g., a barcode sequence) present in the sample. In some embodiments, the analysis comprises quantification of puncta (e.g., if amplification products are detected). In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some cases, the analysis further comprises displaying the information from the analysis or detection step. In some embodiments, software may be used to automate the processing, analysis, and/or display of data.

In some embodiments, the nucleic acid complex is a first nucleic acid complex, the target nucleic acid is a first target nucleic acid, the target sequence is a first target sequence, the detectable label is a first detectable label, the photosensitizer is a first photosensitizer, the target-binding region is a first target-binding region, and the signal is a first signal. In some embodiments, the method further comprises: a′) forming a second nucleic acid complex in the biological sample, wherein the biological sample comprises a second target nucleic acid comprising a second target sequence, and wherein the second nucleic acid complex comprises a second detectable label, a second photosensitizer, and a second target-binding region hybridized to the second target sequence; b′) detecting a second signal associated with the second detectable label of the second nucleic acid complex; and c′) activating the second photosensitizer of the second nucleic acid complex, thereby attenuating the second signal. In some embodiments, the first target nucleic acid and the second target nucleic acid are the same. In some embodiments, the first target nucleic acid and the second target nucleic acid are different. In some embodiments, the first target sequence and the second target sequence are the same. In some embodiments, the first target nucleic acid and the second target sequence are different. In some embodiments, the first detectable label and the second detectable label are the same. In some embodiments, the first detectable label and the second detectable label are different. In some embodiments, the first photosensitizer and the second photosensitizer are the same. In some embodiments, the first photosensitizer and the second photosensitizer are different. In some embodiments, the first target sequence and the second target sequence are the same, and the first photosensitizer and the second photosensitizer are the same. In some embodiments, the first target sequence and the second target sequence are different, and the first photosensitizer and the second photosensitizer are different.

Exemplary embodiments of the described methods include in situ analysis of nucleic acid amplification products, such as rolling circle amplification (RCA) products (RCPs). For instance, the libraries of nucleic acid complexes for Cycle 1, Cycle 2, and Cycle 3 shown in FIG. 3 can target RCPs in a sample, and the gene-specific barcode in each nucleic acid complex can hybridize to multiple copies of a complementary barcode in an RCP. Thus, in some aspects provided herein is a method for analyzing a biological sample, comprising: a) generating a RCP in the biological sample, the RCP comprising multiple copies of a barcode sequence, wherein the barcode sequence is associated with an analyte of interest (e.g., a target nucleic acid comprising a target sequence) and is assigned a signal code sequence; b) contacting the biological sample with a first probe (e.g., intermediate probe such as an L-probe) and a first probe complex to generate a first nucleic acid complex comprising the first probe hybridized to the RCP and the first probe complex hybridized to the first probe, wherein the first probe comprises (i) a recognition sequence (e.g., a target-binding region) complementary to the barcode sequence and (ii) a first overhang sequence, and wherein the first probe complex comprises a first splint oligonucleotide and a first coupling oligonucleotide hybridized thereto, wherein the first splint oligonucleotide comprises a first detectable label and a sequence complementary to the first overhang sequence, and wherein the first coupling oligonucleotide comprises a first photosensitizer; c) detecting a first signal associated with the first detectable label, wherein the first signal corresponds to a first signal code in the signal code sequence; d) activating the first photosensitizer, thereby attenuating the first signal; e) contacting the biological sample with a second probe (e.g., intermediate probe such as an L-probe) and a second probe complex to generate a second nucleic acid complex comprising the second probe hybridized to the RCA product and the second probe complex hybridized to the second probe, wherein the second probe comprises (i) a recognition sequence (e.g., a target-binding region) complementary to the barcode sequence and (ii) a second overhang sequence, and wherein the second probe complex comprises a second splint oligonucleotide and a second coupling oligonucleotide hybridized thereto, wherein the second splint oligonucleotide comprises a second detectable label and a sequence complementary to the second overhang sequence, and wherein the second coupling oligonucleotide comprises a second photosensitizer; and f) detecting a second signal associated with the second detectable label, wherein the second signal corresponds to a second signal code in the signal code sequence, wherein the signal code sequence comprising the first signal code and the second signal code is determined at a location in the biological sample, thereby decoding the barcode sequence and identifying the analyte of interest at the location in the biological sample. In some embodiments, the method further comprises g) activating the second photosensitizer, thereby attenuating the second signal. In some embodiments, the first detectable label and the second detectable label are the same. In some embodiments, the first detectable label and the second detectable label are different. In some embodiments, the first photosensitizer and the second photosensitizer are the same. In some embodiments, the first photosensitizer and the second photosensitizer are different. In some embodiments, the first target sequence and the second target sequence are the same, and the first photosensitizer and the second photosensitizer are the same. In some embodiments, the first target sequence and the second target sequence are different, and the first photosensitizer and the second photosensitizer are different. In some embodiments, the first signal code and the second signal code are the same. In some embodiments, the first signal code and the second signal code are different.

In some embodiments, the first probe, the second probe, and one or more subsequent probes are contacted with the biological sample sequentially in a pre-determined sequence which corresponds to the signal code sequence assigned to the barcode sequence, wherein the one or more subsequent probes each comprises (i) a recognition sequence complementary to the barcode sequence and (ii) an overhang sequence complementary to a probe complex of a universal pool of probe complexes. In some embodiments, the biological sample is contacted with the first probe before the second probe and one or more subsequent probes. In some embodiments, the biological sample is contacted with the second after the first probe and before and one or more subsequent probes. In some embodiments, the biological sample is contacted with the one or more subsequent probes after the first probe. In some embodiments, the biological sample is contacted with the one or more subsequent probes after the first probe and the second probe.

In some embodiments, the first probe complex and the second probe complex are in the universal pool of probe complexes. In some embodiments, a “universal pool of probe complexes” comprises at least two probe complexes (e.g., a first probe complex and a second probe complex), and may be used for multiplexing analyses of two or more target analytes (e.g., target nucleic acids) in a biological sample. In some embodiments, the contacting of the biological sample with a first probe and a first probe complex (e.g., in step b)) comprises contacting the biological sample with the universal pool of probe complexes, and the contacting of the biological sample with a second probe and a second probe complex (e.g., in e)) comprises contacting the biological sample with the universal pool of probe complexes. In some embodiments, the universal pool of probe complexes used in the contacting in b) is the same as the universal pool of probe complexes used in the contacting in e). In some embodiments, the number of different probe complexes in the universal pool of probe complexes is four, for instance, as shown in FIG. 3.

In some embodiments, the one or more subsequent probes are contacted with the biological sample to determine signal codes in the signal code sequence until sufficient signal codes have been determined to decode the barcode sequence, thereby identifying the target analyte (e.g., target nucleic acid). In some embodiments, the method further comprises between the activating of the first photosensitizer (e.g., step in d)) and the contacting of the biological sample with a second probe and a second probe complex (e.g., in e)), a step of removing the first probe and/or the first probe complex from the biological sample before contacting the sample with a subsequent probe. In some embodiments, the method further comprises after the detecting of the second signal associated with the second detectable label (e.g., step in f)), a step of removing the second probe and/or the second probe complex from the biological sample, before contacting the sample with a subsequent probe. In some embodiments, the method further comprises between the activating of the first photosensitizer and the contacting of the biological sample with a second probe and a second probe complex, a step of removing the first probe and/or the first probe complex from the biological sample, and after the detecting of the second signal, a step of removing the second probe and/or the second probe complex from the biological sample, before contacting the sample with a subsequent probe, before contacting the sample with a subsequent probe.

In some embodiments, the barcode sequence associated with the target analyte is selected from a plurality of barcode sequences. In some embodiments, the contacting of the biological sample with a first probe and a first probe complex (e.g., in step b)) comprises contacting the sample with a first pool of probes and a universal pool of probe complexes, wherein the first pool of probes comprises the first probe and the universal pool of probe complexes comprises the first probe complex and the second probe complex, wherein each probe in the first pool of probes comprises (i) a recognition sequence complementary to one of the plurality of barcode sequences and (ii) an overhang sequence complementary to a probe complex of the universal pool of probe complexes; and wherein the contacting of the biological sample with a second probe and a second probe complex (e.g., in e)) comprises contacting the biological sample with a second pool of probes and the universal pool of probe complexes, wherein the second pool of probes comprises the second probe, and wherein each probe in the second pool of probes comprises (i) a recognition sequence complementary to one of the plurality of barcode sequences and (ii) an overhang sequence complementary to a probe complex of the universal pool of probe complexes. In some embodiments, the method further comprises identifying multiple different target analytes present at locations (e.g., different locations) in the biological sample. In some embodiments, each different target analyte is assigned a different signal code sequence and is targeted by a circularizable probe or probe set comprising a complement of a different barcode sequence of the plurality of barcode sequences. In some embodiments, the number of different probes in each pool of probes is greater than the number of different probe complexes in the universal pool of probe complexes. In some embodiments, the number of different probe complexes in the universal pool of probe complexes is four. In some embodiments, the number of different probes in each pool of probes is about 10, about 20, about 30, about 40, about 50, about 100, about 200, about 500, about 1,000, or more. In some embodiments, the number of different probes in each pool of probes in at least about 10, such as at least any of about 20, 30, 40, 50, 100, 200, 500, 1,000, or more.

In some embodiments, for each nucleic acid complex, the hybridization between the nucleic acid probe and the splint oligonucleotide has a higher melting temperature than the hybridization between the nucleic acid probe and the target sequence. In some embodiments, the hybridization between the nucleic acid probe and the splint oligonucleotide has at least about 0.5° C. higher melting temperature than the hybridization between the nucleic acid probe and the target sequence, such as at least any of about 1° C., 2° C., 3° C., 4° C., 5° C., 6° C., 7° C., 8° C., 9° C., 10° C., or more, higher melting temperature than the hybridization between the nucleic acid probe and the target sequence. The higher melting temperature may confer increased stability of the hybridization between the nucleic acid probe and the splint oligonucleotide compared to that of the nucleic acid probe and the target sequence. Therefore, the nucleic acid probe and the splint oligonucleotide of the nucleic acid complex may remain hybridized under conditions wherein the nucleic acid probe and the target sequence become unhybridized.

### III. Samples, Analytes, and Target Sequences

A. Samples

A sample disclosed herein can be or derived from any biological sample. Methods, probes, and kits disclosed herein may be used for analyzing a biological sample, which may be obtained from a subject using any of a variety of techniques including, but not limited to, biopsy, surgery, and laser capture microscopy (LCM), and generally comprises cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, a cell pellet, a cell block needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe (e.g., a padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015, the content of which is herein incorporated by reference in its entirety.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9×its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel comprises a hybrid material, e.g., the hydrogel material comprises elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate comprises a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. If a sample is not permeabilized sufficiently, probes that enter the sample and bind to analytes therein may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods that can be used herein include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A. et al., cDNA normalization by hydroxyapatite chromatography to enrich transcriptome diversity in RNA-seq applications, Biotechniques, 53(6) 373-80, (2012), the entire contents of which are incorporated herein by reference).

B. Analytes

A biological sample may comprise one or a plurality of analytes of interest. Methods for performing multiplexed assays to analyze two or more different analytes in a single biological sample are provided.

The methods, probes, and kits disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule, macromolecule, or chemical compound, including a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules (e.g., cellular nucleic acids), such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which comprises a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. In some embodiments, provided are methods to analyze analytes in a biological sample comprising cellular nucleic acids. Methods, probes, and kits disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods, probes, and kits disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte can comprise or be associated with a target sequence. In some embodiments, the target nucleic acid and the target sequence therein may be endogenous to the sample, generated in the sample, added to the sample, or associated with an analyte in the sample. In some embodiments, the target sequence is a single-stranded target sequence (e.g., a sequence in a rolling circle amplification product). In some embodiments, the target sequence is a single-stranded target sequence (e.g., in a probe bound directly or indirectly to the analyte). In some embodiments, the target sequence is a single-stranded target sequence in a primary probe that binds to an analyte of interest in the biological sample. In some embodiments, the target sequence is a single-stranded target sequence in an intermediate probe which directly or indirectly binds to a primary probe or product thereof, where the primary probe binds to an analyte of interest (e.g., a cellular nucleic acid) in the biological sample. In some embodiments, the target sequence is a single-stranded target sequence in a secondary probe that binds to the primary probe or product thereof. In some embodiments, the analytes comprises one or more single-stranded target sequences.

### (ii) Labelling Agents

In some embodiments, provided herein are methods, probes, and kits for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. For example, in some embodiments, nucleic acid complexes provided herein may be used for the detection of target sequence(s) in a reporter oligonucleotide. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode comprises to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety comprises one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes comprises a single species of analyte (e.g., a single species of polypeptide). In some embodiments in which the plurality of analytes comprises a single species of analyte, the analyte binding moieties of the plurality of analyte labelling agents are the same. In some embodiments in which the plurality of analytes comprises a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes comprises multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods, probes, and kits for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed. For example, a product comprising a target sequence for a target-binding region in a nucleic acid complex (e.g., as described in Section II.A) may be a hybridization complex formed of a cellular nucleic acid in a sample and an exogenously added nucleic acid probe or a product generated therefrom.

a. Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or an exogenous molecule such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. In some instances, various probes and probe sets can be used to generate a product comprising a target sequence that can be hybridized by a nucleic acid complex described herein. In some instances, a probe or probe set disclosed herein is a circularizable probe or probe set comprising a barcode region comprising one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

b. Ligation

In some embodiments, a product of an endogenous analyte and/or a labelling agent is a ligation product that may comprise a target sequence that can be hybridized by the nucleic acid complexes described herein. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent or probe, for example, the circularization of a circularizable probe or probe set upon hybridization to a target sequence. The target sequence can be comprised in an endogenous analyte (e.g., nucleic acid such as genomic DNA or mRNA) or a product thereof (e.g., cDNA from a cellular mRNA transcript), or in a labelling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety.

In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, a circular or circularizable probe or probe set may be used to analyze a reporter oligonucleotide, which may generated using proximity ligation or be subjected to proximity ligation. In some examples, the reporter oligonucleotide of a labelling agent that specifically recognizes a protein can be analyzed using in situ hybridization (e.g., sequential hybridization) and/or in situ sequencing (e.g., using circular or circularizable probes and rolling circle amplification of circular or circularized probes). Further, the reporter oligonucleotide of the labelling agent and/or a complement thereof and/or a product (e.g., a hybridization product, a ligation product, an extension product (e.g., by a DNA or RNA polymerase), a replication product, a transcription/reverse transcription product, and/or an amplification product) thereof can be recognized by another labelling agent and analyzed.

In some embodiments, an analyte (a nucleic acid analyte or non-nucleic acid analyte) can be specifically bound by two labelling agents (e.g., antibodies) each of which is attached to a reporter oligonucleotide (e.g., DNA) that can participate in ligation, replication, and sequence decoding reactions, e.g., using a probe or probe set (e.g., a circularizable probe, a padlock probe, a SNAIL probe set, a circular probe, a gapped padlock probe, or a gapped padlock probe and a connector). In some embodiments, the probe set may comprise two or more probe oligonucleotides, each comprising a region that is complementary to each other. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods. (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions.

In some embodiments, one or more reporter oligonucleotides (and optionally one or more other nucleic acid molecules such as a connector) aid in the ligation of the probe. Upon ligation, the probe may form a circularized probe. In some embodiments, one or more suitable probes can be used and ligated, wherein the one or more probes comprise a sequence that is complementary to the one or more reporter oligonucleotides (or portion thereof). The probe may comprise one or more barcode sequences. In some embodiments, the one or more reporter oligonucleotide may serve as a primer for rolling circle amplification (RCA) of the circularized probe. In some embodiments, a nucleic acid other than the one or more reporter oligonucleotide is used as a primer for rolling circle amplification (RCA) of the circularized probe. For example, a nucleic acid capable of hybridizing to the circularized probe at a sequence other than sequence(s) hybridizing to the one or more reporter oligonucleotide can be used as the primer for RCA. In other examples, the primer in a SNAIL probe set is used as the primer for RCA.

In some embodiments, one or more analytes can be specifically bound by two primary antibodies, each of which is in turn recognized by a secondary antibody each attached to a reporter oligonucleotide (e.g., DNA). Each nucleic acid molecule can aid in the ligation of the probe to form a circularized probe. In some instances, the probe can comprise one or more barcode sequences. Further, the reporter oligonucleotide may serve as a primer for rolling circle amplification of the circularized probe. The nucleic acid molecules, circularized probes, and RCA products can be analyzed using any suitable method disclosed herein for in situ analysis.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe (e.g., padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

c. Primer Extension

In some embodiments, a product is a primer extension product of an analyte, a labelling agent, a probe or probe set bound to the analyte (e.g., a circularizable probe bound to genomic DNA, mRNA, or cDNA), or a probe or probe set bound to the labelling agent (e.g., a circularizable probe bound to one or more reporter oligonucleotides from the same or different labelling agents). Any of such products of extension may comprise a target sequence that can be hybridized by the nucleic acid complexes described herein.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include linear RCA, a branched RCA, a dendritic RCA, or any combination thereof. See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:e118, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801, all of which are herein incorporated by reference in their entireties. Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2016/0024555, US 2018/0251833 and US20170219465, all of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, the detection of numerous different analytes may use a RCA-based detection system, e.g., where the signal is provided by generating a target sequence from a circular RCA template which is provided or generated in the assay, and the target sequence is detected to detect the corresponding analyte. The target sequence may thus be regarded as a reporter which is detected to detect the target analyte. However, the RCA template may also be regarded as a reporter for the target analyte; the target sequence is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g. a reagent in a detection assay), which is used as a reporter for the assay, or a part of a reporter, or signal-generation system. The RCA template used to generate the target sequence may thus be a circular (e.g. circularized) reporter nucleic acid molecule, namely from any RCA-based detection assay which uses or generates a circular nucleic acid molecule as a reporter for the assay. Since the RCA template generates the target sequence reporter, it may be viewed as part of the reporter system for the assay.

In some embodiments, a product herein comprises a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a target-binding region in a nucleic acid complex may be a hybridization complex formed of a cellular nucleic acid in a sample and an exogenously added nucleic acid probe or a product generated therefrom. The exogenously added nucleic acid probe (e.g., primary probe) may comprise an overhang that does not hybridize to the cellular nucleic acid but hybridizes to another probe (e.g., an intermediate probe) or nucleic acid complexes such as depicted in FIG. 2. In other examples, a product comprising a target sequence for a target-binding region in a nucleic acid complex may be a target sequence of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a target-binding region in a nucleic acid complex may be generated using a probe hybridizing to a cellular nucleic acid.

In some instances, a target nucleic acid comprising a target sequence for a target-binding region in a nucleic acid complex is a product that comprises one or more signal amplification components. In some instances, the amplification comprises one or more probe hybridizations and generation of amplified signals associated with the probes (e.g., primary and/or intermediate probes). In some instances, the target nucleic acid (e.g., primary probe as depicted in the middle panel of FIG. 2) comprises one or more target sequence(s) for nucleic acid complex hybridization, such that the signal is amplified by the presence of multiple nucleic acid complexes hybridized to the target nucleic acid. In some instances, the target nucleic acid (e.g., intermediate probe as depicted in the right panel of FIG. 2) comprises multiple target sequences for nucleic acid complex hybridization, such that the signal corresponding to a barcode sequence is amplified by the presence of multiple nucleic acid complexes hybridized to the target nucleic acid. Exemplary signal amplification methods include targeted assembly of branched structures (e.g., bDNA). In some instances, detection of nucleic acids sequences in situ includes combination of the sequential decoding methods described herein with an assembly for branched signal amplification using the nucleic acid complexes provided herein. In some instances, the assembly complex comprises an amplifier hybridized directly or indirectly (via one or more oligonucleotides) to a sequence of a cellular nucleic acid.

### IV. Systems for Analysis of Biological Samples

Provided is a system for performing the methods (e.g., assays described in Section II) including a source (e.g., light source) for activating the photosensitizer of the nucleic acid complex and/or imaging signals from the sample being assayed. In some embodiments, the system comprises a source for photo-activating the photoreactive nucleotide to covalently couple the splint oligonucleotide and the coupling oligonucleotide. In some embodiments, the source is a light source. Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) using the nucleic acid complexes as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., one or more cycles as described in Section II). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules (e.g., any of the target analytes, labelling agents, or products described in Section III.B) in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample and/or activating the photosensitizer of the nucleic acid complex) and/or imaging light signals received from the probed sample. The in-situ analysis system may also include other ancillary modules configured to facilitate the operation of the opto-fluidic instrument, such as, but not limited to, cooling systems, motion calibration systems, etc.

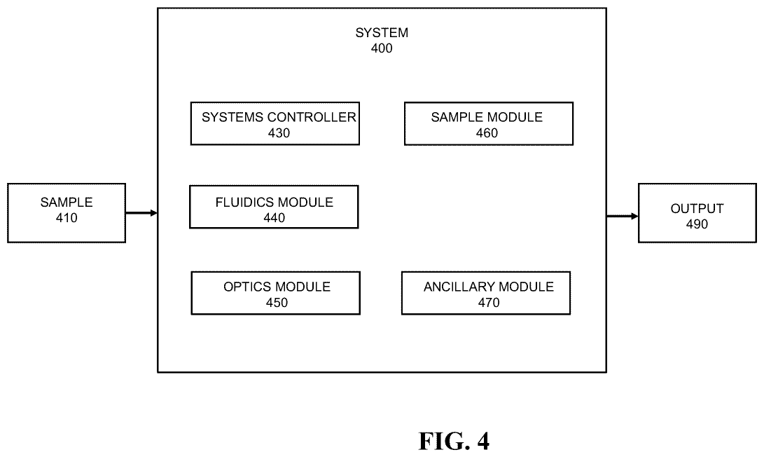

FIG. 4 shows an example workflow of analysis of a biological sample 410 (e.g., cell or tissue sample) using an opto-fluidic instrument 420, according to various embodiments. In various embodiments, the sample 410 can be a biological sample (e.g., a tissue) that includes molecules such as DNA, RNA, proteins, antibodies, etc. For example, the sample 410 can be a sectioned tissue that is treated to access the RNA thereof for labeling with probes described herein (e.g., in Section II). Ligation of a circularizable probe or probe set may generate a circular probe which can be enzymatically amplified and bound with detectably labeled probes, which can create bright signal that is convenient to image and has a high signal-to-noise ratio.

In various embodiments, the sample 410 may be placed in the opto-fluidic instrument 420 for analysis and detection of the molecules in the sample 410. In various embodiments, the opto-fluidic instrument 420 can be a system configured to facilitate the experimental conditions conducive for the detection of the target molecules. For example, the opto-fluidic instrument 420 can include a fluidics module 440, an optics module 450, a sample module 460, and an ancillary module 470, and these modules may be operated by a system controller 430 to create the experimental conditions for the probing of the molecules in the sample 410 by selected probes (e.g., circularizable DNA probes), as well as to facilitate the imaging of the probed sample (e.g., by an imaging system of the optics module 450). In various embodiments, the various modules of the opto-fluidic instrument 420 may be separate components in communication with each other, or at least some of them may be integrated together.

In various embodiments, the sample module 460 may be configured to receive the sample 410 into the opto-fluidic instrument 420. For instance, the sample module 460 may include a sample interface module (SIM) that is configured to receive a sample device (e.g., cassette) onto which the sample 410 can be deposited. That is, the sample 410 may be placed in the opto-fluidic instrument 420 by depositing the sample 410 (e.g., the sectioned tissue) on a sample device that is then inserted into the SIM of the sample module 460. In some instances, the sample module 460 may also include an X-Y stage onto which the SIM is mounted. The X-Y stage may be configured to move the SIM mounted thereon (e.g., and as such the sample device containing the sample 410 inserted therein) in perpendicular directions along the two-dimensional (2D) plane of the opto-fluidic instrument 420.

The experimental conditions that are conducive for the detection of the molecules in the sample 410 may depend on the target molecule detection technique that is employed by the opto-fluidic instrument 420. For example, in various embodiments, the opto-fluidic instrument 420 can be a system that is configured to detect molecules in the sample 410 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 410 using reagents such as washing/stripping reagents, hybridizing reagents, etc., and such reagents may be provided by the fluidics module 440.

In various embodiments, the fluidics module 440 may include one or more components that may be used for storing the reagents, as well as for transporting said reagents to and from the sample device containing the sample 410. For example, the fluidics module 440 may include reservoirs configured to store the reagents, as well as a waste container configured for collecting the reagents (e.g., and other waste) after use by the opto-fluidic instrument 420 to analyze and detect the molecules of the sample 410. Further, the fluidics module 440 may also include pumps, tubes, pipettes, etc., that are configured to facilitate the transport of the reagent to the sample device (e.g., and as such the sample 410). For instance, the fluidics module 440 may include pumps (“reagent pumps”) that are configured to pump washing/stripping reagents to the sample device for use in washing/stripping the sample 410 (e.g., as well as other washing functions such as washing an objective lens of the imaging system of the optics module 450).

In various embodiments, the ancillary module 470 can be a cooling system of the opto-fluidic instrument 420, and the cooling system may include a network of coolant-carrying tubes that are configured to transport coolants to various modules of the opto-fluidic instrument 420 for regulating the temperatures thereof. In such cases, the fluidics module 440 may include coolant reservoirs for storing the coolants and pumps (e.g., “coolant pumps”) for generating a pressure differential, thereby forcing the coolants to flow from the reservoirs to the various modules of the opto-fluidic instrument 420 via the coolant-carrying tubes. In some instances, the fluidics module 440 may include returning coolant reservoirs that may be configured to receive and store returning coolants, i.e., heated coolants flowing back into the returning coolant reservoirs after absorbing heat discharged by the various modules of the opto-fluidic instrument 420. In such cases, the fluidics module 440 may also include cooling fans that are configured to force air (e.g., cool and/or ambient air) into the returning coolant reservoirs to cool the heated coolants stored therein. In some instance, the fluidics module 440 may also include cooling fans that are configured to force air directly into a component of the opto-fluidic instrument 420 so as to cool said component. For example, the fluidics module 440 may include cooling fans that are configured to direct cool or ambient air into the system controller 430 to cool the same.

As discussed above, the opto-fluidic instrument 420 may include an optics module 450 which include the various optical components of the opto-fluidic instrument 420, such as but not limited to a camera, an illumination module (e.g., LEDs), an objective lens, and/or the like. The optics module 450 may include a fluorescence imaging system that is configured to image the fluorescence emitted by the probes (e.g., oligonucleotides) in the sample 410 after the probes are excited by light from the illumination module of the optics module 450.

In some instances, the optics module 450 may also include an optical frame onto which the camera, the illumination module, and/or the X-Y stage of the sample module 460 may be mounted.

In various embodiments, the system controller 430 may be configured to control the operations of the opto-fluidic instrument 420 (e.g., and the operations of one or more modules thereof). In some instances, the system controller 430 may take various forms, including a processor, a single computer (or computer system), or multiple computers in communication with each other. In various embodiments, the system controller 430 may be communicatively coupled with data storage, set of input devices, display system, or a combination thereof. In some cases, some or all of these components may be considered to be part of or otherwise integrated with the system controller 430, may be separate components in communication with each other, or may be integrated together. In other examples, the system controller 430 can be, or may be in communication with, a cloud computing platform.

In various embodiments, the opto-fluidic instrument 420 may analyze the sample 410 and may generate the output 490 that includes indications of the presence of the target molecules in the sample 410. For instance, with respect to the example embodiment discussed above where the opto-fluidic instrument 420 employs a hybridization technique for detecting molecules, the opto-fluidic instrument 420 may cause the sample 410 to undergo successive rounds of detectably labeled probe hybridization (e.g., using two or more sets of fluorescent probes, where each set of fluorescent probes is excited by a different color channel) and be imaged to detect target molecules in the probed sample 410. In such cases, the output 490 may include optical signatures (e.g., a codeword) specific to each gene, which allow the identification of the target molecules.

### V. Compositions and Kits

In some embodiments, disclosed herein is a composition that comprises a nucleic acid complex in a biological sample, e.g., any of the nucleic acid complexes described in Section II. In some embodiments, the complex further comprises a target nucleic acid, e.g., as described in Sections II and III. The nucleic acid complex comprises a detectable label, a photosensitizer, and a target-binding region hybridized to the target sequence.

Also provided herein are kits, for example comprising a plurality of nucleic acid complexes, wherein each nucleic acid complex comprises a detectable label, a photosensitizer, and a target-binding region, e.g., any described in Section II, and instructions for performing the methods provided herein. In some embodiments, each nucleic acid complex comprises the same photosensitizer, one of four or more different detectable labels, and one of four or more different target-binding regions, wherein the detectable label corresponds to the target-binding region, e.g., any described in Section II. In some embodiments, the kits further comprise multiple probes each comprising i) a target sequence complementary to one of the target-binding regions of the plurality of nucleic acid complexes, and ii) a recognition sequence complementary to a nucleic acid molecule, e.g., any described in Section II. In some embodiments, the hybridization between the target sequence and the target-binding region has a higher melting temperature than the hybridization between the recognition sequence and the nucleic acid molecule. In some embodiments, the recognition sequence comprises a barcode sequence or complement thereof corresponding to an analyte of interest. In some embodiments, the plurality of nucleic acid complexes collectively comprise four different detectable labels each corresponding to the target-binding region of a given nucleic acid complex; and the multiple probes collectively comprise at least or about 10, at least or about 25, at least or about 50, at least or about 100, at least or about 250, at least or about 500, at least or about 750, at least or about 1,000, or more different recognition sequences.

In some embodiments, the kits further comprise one or more reagents required for one or more steps comprising hybridization, ligation, extension, detection, sequencing, and/or sample preparation as described herein. In some embodiments, the kit further comprises a target nucleic acid, e.g., any described in Sections II and III. In some embodiments, any or all of the oligonucleotides are DNA molecules. In some embodiments, the target nucleic acid is a messenger RNA molecule. In some embodiments, the target nucleic acid is a probe (e.g., a circularizable probe or padlock probe, such as a molecule for generating a nucleic acid concatemer) or an amplification product thereof (e.g., a nucleic acid concatemer, such as a rolling circle amplification product).

In some embodiments, provided herein is a kit comprising a plurality of nucleic acid complexes, each comprising a barcode region as the target-binding region, and each nucleic acid complex can comprise a photosensitizer and a detectable label. In some embodiments, the barcode regions in the plurality of nucleic acid complexes are configured to combinatorially encode multiple analytes, where the combinations of the presence or absence of barcodes in the primary probes can be used to encode the multiple analytes. In some embodiments, the plurality of nucleic acid complexes share the same photosensitizer (e.g., a common or universal photosensitizer) and two or more nucleic acid complexes can comprise different detectable label. In some embodiments, two or more nucleic acid complexes targeting the same analyte (e.g., the nucleic acid complexes comprising the same target-binding region) may comprise the same photosensitizer or different photosensitizers. In some embodiments, two or more nucleic acid complexes each targeting a different analyte (e.g., the nucleic acid complexes each comprising a different target-binding region) may comprise the same photosensitizer or different photosensitizers.

In some embodiments, provided herein is a kit comprising a library of nucleic acid complexes, each comprising a target-binding region (e.g., a target-specific barcode) for a different analyte. In some embodiments, the target-specific barcode is used to identify the corresponding target analyte. In some embodiments, the target-specific barcode is a gene-specific barcode that can be used to identify a gene or a transcript thereof. In some embodiments, provided herein is a kit comprising multiple libraries of nucleic acid complexes, for use to combinatorially detect a plurality of analytes at one or more locations in a biological sample. The target-specific barcode in each detectable probe can bind to a sequence (e.g., a complementary barcode sequence) in an RCA product (e.g., as shown in FIG. 2, left panel), a sequence in a primary probe (e.g., as shown in FIG. 2, middle panel), or a sequence in an intermediate probe (e.g., as shown in FIG. 2, right panel) which may be in a branched hybridization complex. In some embodiments, the kit further comprises one or more circular or circularizable probes or probe sets for generating the RCA product, one or more primary probes, and/or one or more intermediate probes that form the branched hybridization complex.

In some embodiments, the kit further comprises probe complexes each comprising one or more splint oligonucleotides and/or one or more coupling oligonucleotides. In some embodiments, the probe complexes are detectably labeled and each probe complex comprises a fluorescently labeled splint oligonucleotide that functions as a detection oligonucleotide (DO) and is configured to hybridize to a nucleic acid probe (e.g., an L-probe or a U-probe) comprising a complement of the DO sequence. In some embodiments, the detectably labeled complex further comprises a coupling oligonucleotide comprising a photosensitizer configured to attenuate a signal of the fluorescent label of the splint oligonucleotide. Each DO sequence can correspond to a different “color,” for example, DO1 to blue (“B”), DO2 to green (“G”), DO3 to yellow (“Y”), and DO4 to red (“R”), as shown in FIG. 3. In some embodiments, the probe complexes form a universal pool of probe complexes that collectively comprise no more than about 10, no more than about 8, no more than about 6, or no more than about 4 different DO sequences each corresponding to a different “color,” and the universal pool of probe complexes can be contacted with a sample in sequential cycles to detect and decode different target-binding regions (e.g., target-specific barcodes).

In some embodiments, the number of different target-binding regions (e.g., target-specific barcodes) in a library of nucleic acid complexes exceeds the number of different DO sequences in the library of nucleic acid complexes. In some embodiments, the number of different target-binding regions is at least about 10, at least about 20, at least about 50, at least about 100, at least about 200, at least about 500, at least about 1,000, at least about 2,000, or at least about 5,000, or more, while the number of different DO sequences is no more than about 6, e.g., the library of nucleic acid complexes can comprise 4 different DO sequences each corresponding to a different “color.”

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers. In some aspects, the kit can also comprise any of the reagents described herein, e.g., wash buffer. In some embodiments, the kits contain reagents for detection (e.g., sequence detection), such as barcode detection probes or detectable labels (e.g., as part of nucleic acid complexes). In some embodiments, the kits optionally contain other components, for example nucleic acid primers, enzymes and reagents, buffers, nucleotides, and reagents for additional assays.

### VI. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to detect a signal associated with a detectable label of a nucleic acid complex that is hybridized to a target sequence of a target nucleic acid in a biological sample. The nucleic acid complexes of the present invention may eliminate punctate nuclear background as well as reducing residual background noise across decoding cycles during in situ analysis.

In some embodiments, the target nucleic acid comprises a single-nucleotide polymorphism (SNP). In some embodiments, the target nucleic acid comprises is a single-nucleotide variant (SNV). In some embodiments, the target nucleic acid comprises a single-nucleotide substitution. In some embodiments, the target nucleic acid comprises a point mutation. In some embodiments, the target nucleic acid comprises a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE or other suitable buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985), the content of which is herein incorporated by reference in its entirety). Other references (e.g., Allawi and SantaLucia, Jr., Biochemistry, 36:10581-94 (1997), the content of which is herein incorporated by reference in its entirety) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999), both of which are herein incorporated by reference in their entireties.

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984), the content of which is herein incorporated by reference in its entirety.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. In some embodiments, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif; HeliScope™ by Helicos Biosciences Corporation, Cambridge, Ma.; and PacBio RS by Pacific Biosciences of California, Inc., Menlo Park, Calif), sequencing by ion detection technologies (such as Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following example is included for illustrative purposes only and not intended to limit the scope of the present disclosure.

### Example 1: Design and Use of Nucleic Acid Complexes Comprising Detectable Labels and Photosensitizers in Proximity

This Example describes the design and use of a nucleic acid complex comprising a splint oligonucleotide and a coupling oligonucleotide. The splint oligonucleotide comprises a detectable label, such as a fluorophore. The detectable label can be linked to the 3′ terminal nucleotide, the 5′ terminal nucleotide, or an internal nucleotide in the splint oligonucleotide. For illustrative purposes only, in the description below the detectable label is at the 3′ end of the splint oligonucleotide. The coupling oligonucleotide can comprise a plurality of functional moieties including a photoreactive nucleotide (e.g., 3-cyanovinylcarbazole nucleoside (^{CNV}K) amidites) so that the coupling oligonucleotide can be covalently attached to the splint oligonucleotide after activation of the photoreactive nucleotide to react with a nucleotide in the splint oligonucleotide hybridized to the coupling oligonucleotide. The coupling oligonucleotide can also comprise a flexible linker attached to biotin on the 5′ end.

When ^{CNV}K is incorporated into an oligonucleotide, rapid photo crosslinking to pyrimidines in the complementary strand can be induced by a specific wavelength, e.g., using a light having a wavelength of 350-400 nm. In some embodiments, the photo crosslinking reaction occurs (e.g., in a container) prior to adding to a sample (e.g., a tissue section) and imaging. The crosslinking of the oligonucleotides (e.g., a splint oligonucleotide and a coupling oligonucleotide disclosed herein) and assembly of an oligonucleotide (e.g., a splint oligonucleotide or a coupling oligonucleotide disclosed herein) with a photosensitizer or a detectable label can occur independently and before contacting the sample. In addition, the crosslinking of the oligonucleotides and the assembly of an oligonucleotide with a photosensitizer can occur before photosensitizer activation that attenuates a signal from the detectable label. Once cross-linked, the UV melting temperature of the duplex is raised by around 30° C./^{CNV}K moiety relative to the duplex before irradiation.

FIG. 1F shows steps that can be used to couple one or more coupling oligonucleotides to the splint oligonucleotide using cross-linking and/or chemical or enzymatic ligation, e.g., by a ligase. A coupling oligonucleotide can comprise a photosensitizer attached at the 5′ end and hybridizes to the 3′ end of the splint oligonucleotide, and upon hybridization, the photosensitizer is brought into proximity to the detectable label. In some cases, since the coupling oligonucleotide can be added after ^{CNV}K irradiation, the photosensitizer is not activated by ^{CNV}K irradiation such that it cannot destroy the fluorophore. In other cases, infrared photosensitizers (e.g., activated using light of about 800 nm and about 1 mm in wavelength) can be used, such that the infrared photosensitizers are not activated during ^{CNV}K irradiation (e.g., using wavelength of 350-400 nm) even when the photosensitizer is in proximity to the detectable label during ^{CNV}K irradiation.

Photosensitizers are molecules that when excited with a specific wavelength emit reactive oxygen species (ROS) in the form of singlet oxygen, which can decay with a radius of 1/r⁴ and so only molecules in close proximity are damaged by the ROS. Phthalocyanine derivatives may be used as photosensitizers herein as they only function with light in the near infrared spectrum (e.g., between about 800 nm and about 2,500 nm).

Following generation, the coupled oligonucleotides can be contacted with a nucleic acid probe that hybridizes to the splint oligonucleotide, as shown in FIGS. 1A-1E, to form a nucleic acid complex which is then contacted with target nucleic acids in a biological sample. Alternatively, the coupled oligonucleotides and the nucleic acid probe can be contacted with the sample separately, either simultaneously or sequentially in either order, and the nucleic acid complex can be formed in situ in the sample.

Signals associated with the detectable label in nucleic acid complexes contacted with target nucleic acids in a biological sample can be detected, and then the photosensitizer can be activated (e.g., using light of about 800 nm and about 1 mm in wavelength) to attenuate the signal. Streptavidin-conjugated microparticles can optionally be used to capture the biotin moieties in the nucleic acid complexes to facilitate their removal from the sample, before additional coupled oligonucleotides or nucleic acid complexes are contacted with the sample for signal detection, signal attenuation, and nucleic acid complex removal. A first signal associated with the first detectable labeled of a first nucleic acid complex can be detected and it corresponds to a first signal code in the signal code sequence, and subsequent nucleic acid complexes are contacted with the biological sample to determine additional signal codes in the signal code sequence associated with the barcode sequence, thereby identifying the target analyte. In this way, signals associated with the detectable labels of the plurality of nucleic acid complexes is used for a temporal combination or pattern of the “colors” that can be detected and used to decode the gene-specific barcodes at one or more locations in the sample.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for analyzing a biological sample, comprising:
a) forming a nucleic acid complex in the biological sample,
wherein the biological sample comprises a target nucleic acid comprising a target sequence, and
wherein the nucleic acid complex comprises a detectable label, a photosensitizer, and a target-binding region hybridized to the target sequence;
b) detecting a signal associated with the detectable label of the nucleic acid complex; and
c) activating the photosensitizer of the nucleic acid complex, thereby attenuating the signal associated with the detectable label.

2-3. (canceled)

4. The method of claim 1, wherein the nucleic acid complex comprises a nucleic acid probe and a splint oligonucleotide hybridized thereto, and wherein the nucleic acid probe comprises the target-binding region, and wherein:
(i) the nucleic acid probe comprises the photosensitizer and the splint oligonucleotide comprises the detectable label; or
(ii) the nucleic acid probe comprises the detectable label and the splint oligonucleotide comprises the photosensitizer.

5. (canceled)

6. The method of claim 4, wherein the nucleic acid complex further comprises a coupling oligonucleotide that hybridizes to the splint oligonucleotide and/or the nucleic acid probe.

7-8. (canceled)

9. The method of claim 6, wherein:
the splint oligonucleotide comprises the detectable label and the coupling oligonucleotide comprises the photosensitizer; or
the splint oligonucleotide comprises the photosensitizer and the coupling oligonucleotide comprises the detectable label.

10. (canceled)

11. The method of claim 1 wherein the splint oligonucleotide and the coupling oligonucleotide are configured to covalently couple to each other.

12. The method of claim 11, wherein the splint oligonucleotide and/or the coupling oligonucleotide each independently comprises a photoreactive nucleotide capable of reacting with a nucleotide in an oligonucleotide strand hybridized thereto to form a covalent bond.

13. The method of claim 12, wherein the photoreactive nucleotide comprises a 3-cyanovinylcarbazole (^{CNV}K) nucleoside, 3-cyanovinylcarbazole modified D-threoninol (^{CNV}D), or 3-cyanovinylcarbazole phosphoramidite.

14. The method of claim 12, comprising photo-activating the photoreactive nucleotide to covalently couple the splint oligonucleotide and the coupling oligonucleotide, wherein the photo-activating is performed prior to the splint oligonucleotide and the coupling oligonucleotide contacting the biological sample and/or wherein the photo-activating is performed without activating the photosensitizer.

15-17. (canceled)

18. The method of claim 6, wherein the splint oligonucleotide comprises the detectable label, and the method comprises:
contacting the splint oligonucleotide and the coupling oligonucleotide with an oligonucleotide comprising the photosensitizer;
ligating the oligonucleotide comprising the photosensitizer to the coupling oligonucleotide using the splint oligonucleotide as a template, whereby the photosensitizer and the detectable label are brought into proximity with each other.

19. (canceled)

20. The method of claim 6, wherein:
(i) any two or all of the nucleic acid probe, the splint oligonucleotide, and the coupling oligonucleotide form a complex prior to contacting with the biological sample, or (ii) any two or all of the nucleic acid probe, the splint oligonucleotide, and the coupling oligonucleotide form a complex in the biological sample.

21-27. (canceled)

28. The method of claim 1, wherein the photosensitizer is activated using an infrared light or near-infrared light.

29-33. (canceled)

34. The method of claim 1, wherein activation of the photosensitizer generates a reactive oxygen species, wherein the reactive oxygen species reacts with the detectable label to render it undetectable under the conditions for the detecting the signal associated with the detectable label.

35-41. (canceled)

42. The method of claim 1, wherein the nucleic acid complex further comprises an affinity moiety.

43-49. (canceled)

50. The method of claim 1, wherein the nucleic acid complex further comprises a reflective moiety.

51-54. (canceled)

55. The method of claim 1, wherein the signal associated with the detectable label is detected in situ and the photosensitizer is activated in situ.

56. The method of claim 1, wherein the target nucleic acid comprises or is:
i) a cellular nucleic acid molecule or a product thereof,
ii) a primary probe that hybridizes to a cellular nucleic acid molecule or a product thereof, or
iii) an intermediate probe that hybridizes to a primary probe or a product or complex thereof, wherein the primary probe hybridizes to a cellular nucleic acid molecule or a product thereof.

57-63. (canceled)

64. The method of claim 56, wherein the product or complex of the primary probe is selected from the group consisting of:
a rolling circle amplification (RCA) product,
a complex comprising an initiator and an amplifier for hybridization chain reaction (HCR),
a complex comprising an initiator and an amplifier for linear oligonucleotide hybridization chain reaction (LO-HCR),
a primer exchange reaction (PER) product, and
a complex comprising a pre-amplifier and an amplifier for branched DNA (bDNA).

65-68. (canceled)

69. The method of claim 1, wherein:
the target nucleic acid comprises an overhang region comprising multiple copies of the target sequence; or
the target nucleic acid is concatemeric and comprises multiple copies of the target sequence.

70. (canceled)

71. The method of claim 1, further comprising
generating in situ in the biological sample the target nucleic acid or a molecule or complex to which the target nucleic acid directly or indirectly binds.

72-78. (canceled)

79. A method for analyzing a biological sample, comprising:
a) generating a rolling circle amplification (RCA) product in the biological sample, the RCA product comprising multiple copies of a barcode sequence, wherein the barcode sequence is associated with an analyte of interest and is assigned a signal code sequence;
b) contacting the biological sample with a first probe and a first probe complex to generate a first nucleic acid complex comprising the first probe hybridized to the RCA product and the first probe complex hybridized to the first probe,
wherein the first probe comprises (i) a recognition sequence complementary to the barcode sequence and (ii) a first overhang sequence, and
wherein the first probe complex comprises a first splint oligonucleotide and a first coupling oligonucleotide hybridized thereto, wherein the first splint oligonucleotide comprises a first detectable label and a sequence complementary to the first overhang sequence, and wherein the first coupling oligonucleotide comprises a first photosensitizer;
c) detecting a first signal associated with the first detectable label, wherein the first signal corresponds to a first signal code in the signal code sequence;
d) activating the first photosensitizer, thereby attenuating the first signal;
e) contacting the biological sample with a second probe and a second probe complex to generate a second nucleic acid complex comprising the second probe hybridized to the RCA product and the second probe complex hybridized to the second probe,
wherein the second probe comprises (i) a recognition sequence complementary to the barcode sequence and (ii) a second overhang sequence, and
wherein the second probe complex comprises a second splint oligonucleotide and a second coupling oligonucleotide hybridized thereto, wherein the second splint oligonucleotide comprises a second detectable label and a sequence complementary to the second overhang sequence, and wherein the second coupling oligonucleotide comprises a second photosensitizer; and
f) detecting a second signal associated with the second detectable label, wherein the second signal corresponds to a second signal code in the signal code sequence,
wherein the signal code sequence comprising the first signal code and the second signal code is determined at a location in the biological sample, thereby decoding the barcode sequence and identifying the analyte of interest at the location in the biological sample.

80-97. (canceled)
